(19) United States
(12) Patent Application Publication
(10) Pub. No.: US 2024/0299582 A1
(43) Pub. Date: Sep. 12, 2024

(54) VIRAL DELIVERY OF A SIALIDASE TO TREAT CANCER

(71) Applicant: UNIVERSITÄT BASEL, Basel (CH)

(72) Inventors: Heinz LÄUBLI, Binningen (CH); Natalia RODRIGUES MANTUANO, Basel (CH)

(73) Assignee: UNIVERSITÄT BASEL, Basel (CH)

(21) Appl. No.: 18/546,795

(22) PCT Filed: Feb. 18, 2022
(86) PCT No.: PCT/EP2022/054067

§ 371 Date: Aug. 17, 2023

(30) Foreign Application Priority Data
Feb. 18, 2021 (EP) 21157979.2
Jun. 29, 2021 (EP) 21182465.1

(51) Int. Cl. A61K 48/00; A61K 9/00; A61K 38/47; A61P 35/00; C12N 7/00; C12N 15/86

(52) U.S. Cl. CPC A61K 48/005; A61K 9/0019; A61K 38/47; A61K 48/0075; A61P 35/00; C12N 7/00; C12N 15/86; C12Y 302/01018; C12N 2750/14122; C12N 2750/14143; C12N 2760/16233

(57) ABSTRACT

The present invention relates to adenovirus-associated virus comprising an influenza-derived neuraminidase transgene, used alone or together with an immune checkpoint inhibitor to treat a patient diagnosed with a solid cancer.

The present invention relates to an adeno-associated virus bearing a sialidase gene for use in treating cancer.

This application claims the benefit of European patent applications EP21157979.2, filed 18 Feb. 2021, and EP21182465.1, filed 29 Jun. 2021, both of which are fully incorporated herein by reference.

### BACKGROUND OF THE INVENTION

Therapeutic desialylation of the tumour microenvironment can lead to strong immune-mediated growth reduction in cancer. Sialidases, also called neuraminidases, are enzymes found in a large number of organisms. Viral sialidases are some of the most well-studied sialidase structures.

Intratumoural injection of viruses bearing a transgene, for example, producing immune-stimulating cytokines is an approved treatment option for patients with advanced melanoma, and is currently being explored in different cancer types including head and neck tumours, triple-negative breast cancer, and cutaneous lymphomas.

Based on the above-mentioned state of the art, the objective of the present invention is to provide means and methods to treat cancer, or to supplement treatments for solid cancer, with administration of a sialidase. This objective is attained by the subject-matter of the independent claims of the present specification, with further advantageous embodiments described in the dependent claims, examples, figures and general description of this specification.

### SUMMARY OF THE INVENTION

The present invention relates to an adeno-associated virus (AAV) vector, comprising a sialidase (Sia) transgene, particularly encoding an influenza-derived neuraminidase protein. Further aspects of the invention relate to the use of said adeno-associated virus bearing a neuraminidase (AAV-Sia) as a medicament to treat cancer, or to enhance the effect of an immunotherapy administered to fight cancer, in addition to routes and methods of administration of particular relevance to the invention.

In another embodiment, the present invention relates a pharmaceutical composition comprising the AAV-Sia of the present invention and at least one pharmaceutically acceptable carrier, diluent or excipient, optionally further comprising a checkpoint inhibitor agent.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of an RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

The abbreviation AAV in the context of the present specification relates to adeno-associated virus, a small non-pathogenic virus that infects humans and other primate species. The term AAV is synonymous with AAV virion and AAV viral particle and relates to a viral particle composed of at least one AAV capsid protein and an encapsidated AAV nucleic acid. AAV preparations used for gene delivery purposes are generally reliant of coinfection with a helper virus such as adenovirus in order to replicate, but replication-competent AAV may also be present. Unless otherwise stated, AAV refers to all subtypes or serotypes and both replication-competent and recombinant forms. The term AAV encompasses wild type serotypes such as the serotypes AAV1, 2, 3, 4, 5, 6, 7, 8, 9, 10, and synthetic variants with specific tissue-targeting preferences.

The term transgene in the context of the present specification relates to a gene or genetic material that has been transferred from one organism to another, referring here to the insertion of the neuraminidase gene within the AAV vector. According to embodiments of the invention, this term encompasses an ORF that encodes a neuraminidase derived from the RNA genome of the influenza virus. The skilled person is able to construct a DNA genome adeno viral vector on the basis of this information to make a DNA sequence which encodes the influenza neuraminidase protein. In the present context, the term may also refer to transfer of the expression of a genetic sequence into tissue of a patient infected with the AAV vector.

The term sialidase is used interchangeably with the term neuraminidase in the present specification. The term sialidase or neuraminidase in the context of the present invention refers to the family of glycoside hydrolase enzymes which catalyze the cleavage of non-reducing sialic acid residues, a derivative of neuraminic acid groups, linked to oligosaccharide chains.

The term sialidase derived from an influenza virus in the context of the present invention refers to one of the known variants of the neuraminidase protein naturally occurring in the influenza viruses of the Orthomyxoviridae family.

In the context of the present specification, the term checkpoint inhibitory agent, immune checkpoint inhibitor agent or checkpoint inhibitory antibody is meant to encompass an agent, particularly an antibody (or antibody-like molecule) capable of disrupting the signal cascade leading to T cell inhibition after T cell activation as part of what is known in the art as the immune checkpoint mechanism. Non-limiting examples of a checkpoint inhibitory agent or checkpoint inhibitory antibody include antibodies which inhibit interactions of CTLA-4 (Uniprot P16410), PD-1 (Uniprot Q15116), PD-L1 (Uniprot Q9NZQ7), or B7H3 (CD276; Uniprot Q5ZPR3) with their natural ligands.

In the context of the present specification, the term antibody refers to whole antibodies including but not limited to immunoglobulin type G (IgG), type A (IgA), type D (IgD), type E (IgE) or type M (IgM), any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region of IgG is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. The term encompasses antibody-like molecules such as a so-called nanobody or single domain antibody, an antibody fragment comprising, or consisting of a single monomeric variable antibody domain, or single variable chain fragment (ScFv) multimers.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, dyes, and the like and combinations thereof, as would be known to those skilled in the art (see, for example, Remington: the Science and Practice of Pharmacy, ISBN 0857110624).

As used herein, the term treating or treatment of any disease or disorder (e.g., cancer) refers in one embodiment, to ameliorating the disease or disorder (e.g., slowing or arresting or reducing the development, spread, or the growth rate of a malignant tumour disease). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

### DETAILED DESCRIPTION OF THE INVENTION

A first aspect of the infection relates to a recombinant AAV vector, comprising a transgene encoding an influenza-derived sialidase polypeptide. The term “AAV-Sia” is used synonymously with the term “recombinant AAV vector, comprising a transgene encoding an influenza-derived sialidase polypeptide” throughout this specification. Recombinant in this sense refers to an altered AAV vector comprising the influenza-derived sialidase transgene.

### Sialidase Polypeptides According to the Invention

Certain embodiments of the invention relate to an AAV comprising a sialidase transgene encoding an influenza-derived neuraminidase polypeptide. Known variants of influenza neuraminidase polypeptides (listed in the Uniprot knowledge database under ID: UniProtKB 2021_01) are considered to be feasible alternatives to the Type A neuraminidase 1 (N1) incorporated into the AAV tested in the examples, provided they have similar biological sialidase activity.

In some embodiments, the AAV encodes a sialidase for which broad immunogenicity exists in the human population due to endemic infections and immunization programs. In alternative embodiments, the sialidase is derived from an influenza strain which more commonly infects a different mammalian host (See representative examples in Table 1). This is expected to direct existing immunity derived from vaccination or natural infection against AAV-Sia transduced tumour cells. Additional features such as immunogenicity, nucleic acid length (single-strand AAV vectors are suited to transgenes under 4.4 Kb, while double-strand capacity is half that), and suitability for expression in a human cell may be considered for choosing a suitable neuraminidase transgene.

In some embodiments of the AAV-Sia according to the invention, the sialidase transgene encodes a Type A influenza polypeptide with equivalent biological sialidase function in the context of a recombinant AAV-Sia as the polypeptide designated SEQ ID NO 001.

Sialidase activity of the transgene may be measured in an assay to determine the level of desialylation of tumour cells following transduction with AAV-Sia, such as a test measuring the presence of fluorescent lectins following exposure of tumour cell lines to AAV-Sia as demonstrated in FIG. 2 of the examples. Induction of the sialidase transgene expression in tumour cells transduced with the AAV-Sia vector cleaves sialic acid residues from the tumour cell surface, increasing the binding of peanut agglutinin (PNA) lectin as a detection agent. Desialylation can be defined as an increase in PNA fluorescence intensity on AAV-Sia transduced tumour cells lines of approximately 99% (B16D5), 30% (EMT6), 72% (MC38) and 43% Hela at the viral dose of 2×10⁵ viral particles per target cell, compared to control vector lacking sialidase transgene, or untransduced cells (FIG. 2, dashed line). Sialic acid removal may also be assessed by SNA or MAA staining. Measuring the biological activity according to the invention is defined in the section entitled Biological activity of an AAV-Sia according to the invention below.

In certain embodiments of the AAV-SIa, the sialidase transgene encodes an N1, N2, N3, N4, N5, N6. N7, N8, N9, N10, or N11 neuraminidase (See representative examples in Table 1).

In some embodiments of the AAV-SIa, the sialidase transgene is an N2 influenza neuraminidase. In other embodiments, the sialidase transgene is an N3 influenza neuraminidase. In other embodiments, the sialidase transgene is an N4 influenza neuraminidase. In other embodiments, the sialidase transgene is an N5 influenza neuraminidase. In other embodiments, the sialidase transgene is an N6 influenza neuraminidase. In other embodiments, the sialidase transgene is an N7 influenza neuraminidase. In other embodiments, the sialidase transgene is an N8 influenza neuraminidase. In other embodiments, the sialidase transgene is an N9 influenza neuraminidase. In other embodiments, the sialidase transgene is an N10 influenza neuraminidase. In other embodiments, the sialidase transgene is an N11 influenza neuraminidase. In particular embodiments, the AAV-Sia sialidase transgene encodes an influenza Type A N1 neuraminidase polypeptide.

In alternative embodiments of the AAV-SIa, the sialidase transgene encodes a Type B Victoria lineage neuraminidase. In yet other embodiments, the sialidase transgene encodes a Type B Yamagata lineage neuraminidase.

In other embodiments of the AAV-SIa, the AAV transgene encodes a Type A influenza neuraminidase polypeptide which comprises, or consists of a polypeptide designated SEQ ID NO 002. In other embodiments, the AAV transgene encodes a neuraminidase polypeptide which comprises, or consists of a polypeptide designated SEQ ID NO 003. In other embodiments, the AAV transgene encodes a neuraminidase polypeptide which comprises, or consists of a polypeptide designated SEQ ID NO 004. In other embodiments, the AAV transgene encodes a neuraminidase polypeptide which comprises, or consists of a polypeptide designated SEQ ID NO 005. In other embodiments, the AAV transgene encodes a neuraminidase polypeptide which comprises, or consists of a polypeptide designated SEQ ID NO 006. In other embodiments, the AAV transgene encodes a neuraminidase polypeptide which comprises, or consists of a polypeptide designated SEQ ID NO 007. In other embodiments, the AAV transgene encodes a neuraminidase polypeptide which comprises, or consists of a polypeptide designated SEQ ID NO 008. In other embodiments, the AAV transgene encodes a neuraminidase polypeptide which comprises, or consists of a polypeptide designated SEQ ID NO 009. In other embodiments, the AAV transgene encodes a neuraminidase polypeptide which comprises, or consists of a polypeptide designated or SEQ ID NO 010. These polypeptide sequences encode 9 common Type A-derived polypeptides (SEQ ID NO 001-009) and a Type B-derived neuraminidase polypeptide (SEQ ID NO 010). In particular embodiments demonstrated to be effective to counter tumour growth in the examples, the AAV vector encodes an influenza Type A neuraminidase 1 with the sequence SEQ ID NO 001.

In alternative embodiments, the AAV vector sialidase transgene encodes a polypeptide which comprises, or consists of a polypeptide sequence having an identity of ≥85%, particularly ≥90%, ≥91%, ≥92%, ≥93%, ≥94%, more particularly ≥95%, ≥96%, ≥97%, ≥98%, ≥99%, compared to SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010, provided the sialidase has at least 90% of the biological activity of the specified sialidase polypeptide sequence on which it is based.

In particular alternative embodiments, the AAV vector sialidase transgene encodes a variant polypeptide which comprises, or consists of a polypeptide sequence having an identity of ≥85%, particularly ≥90%, more particularly ≥95% compared to SEQ ID NO 001, and at least 90% of the biological activity of an AAV vector-expressed sialidase polypeptide sequence SEQ ID NO 001.

### Nucleic Acids Sequences of Sialidase AAV Transgenes According to the Invention

In particular embodiments, the AAV-Sia sialidase transgene comprises, a nucleic acid sequence encoding an N1 neuraminidase with the polypeptide sequence SEQ ID NO 001. In further particular embodiments, the AAV-Sia sialidase transgene consists of the nucleic acid sequence encoding an N1 neuraminidase with the polypeptide sequence SEQ ID NO 001.

In other embodiments, the AAV-Sia sialidase transgene comprises, or consists, or consists of a nucleic acid sequence encoding an influenza neuraminidase with the polypeptide sequence SEQ ID NO 002. In other embodiments, the AAV-Sia sialidase transgene comprises, or consists of, a nucleic acid sequence encoding an influenza neuraminidase with the polypeptide sequence SEQ ID NO 003. In other embodiments, the AAV-Sia sialidase transgene comprises, or consists of a nucleic acid sequence encoding an influenza neuraminidase with the polypeptide sequence SEQ ID NO 004. In other embodiments, the AAV-Sia sialidase transgene comprises, or consists of a nucleic acid sequence encoding an influenza neuraminidase with the polypeptide sequence SEQ ID NO 005. In other embodiments, the AAV-Sia sialidase transgene comprises, or consists of a nucleic acid sequence encoding an influenza neuraminidase with the polypeptide sequence SEQ ID NO 006. In other embodiments, the AAV-Sia sialidase transgene comprises, or consists of a nucleic acid sequence encoding an influenza neuraminidase with the polypeptide sequence SEQ ID NO 007. In other embodiments, the AAV-Sia sialidase transgene comprises, or consists of a nucleic acid sequence encoding an influenza neuraminidase with the polypeptide sequence SEQ ID NO 008. In other embodiments, the AAV-Sia sialidase transgene comprises, or consists of a nucleic acid sequence encoding an influenza neuraminidase with the polypeptide sequence SEQ ID NO 009. In other embodiments, the AAV-Sia sialidase transgene comprises, or consists of a nucleic acid sequence encoding an influenza neuraminidase with the polypeptide sequence SEQ ID NO 010.

In particular embodiments, the AAV-Sia carries a transgene comprising the sequence designated SEQ ID NO 011. In alternative embodiments, the AAV-Sia sialidase transgene has the sequence designated SEQ ID NO 012. In further embodiments, the AAV-Sia sialidase transgene has the sequence designated SEQ ID NO 013. In further embodiments, the AAV-Sia sialidase transgene has the sequence designated SEQ ID NO 014. In further embodiments, the AAV-Sia sialidase transgene has the sequence designated SEQ ID NO 015. In further embodiments, the AAV-Sia sialidase transgene has the sequence designated SEQ ID NO 016. In further embodiments, the AAV-Sia sialidase transgene has the sequence designated SEQ ID NO 017. In further embodiments, the AAV-Sia sialidase transgene has the sequence designated SEQ ID NO 018. In further embodiments, the AAV-Sia sialidase transgene has the sequence designated SEQ ID NO 019. In further embodiments, the AAV-Sia sialidase transgene has the sequence designated SEQ ID NO 020.

In particular embodiments, the AAV-Sia sialidase transgene consists of the nucleic acid sequence SEQ ID NO 011.

Alternative embodiments provide an AAV2 vector comprising a sialidase transgene encoding a polypeptide which comprises, or consists of a variant sialidase polypeptide sequence having a sequence identity of ≥85%, particularly ≥90%, more particularly ≥95% compared to an amino acid sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010. According to these embodiments the sialidase has at least 90% of the biological activity of SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010. In particular embodiments, the variant has at least 90% of the biological function of the polypeptide SEQ ID NO 001. In particular embodiments the AAV2 vector comprises a sialidase transgene encoding a polypeptide which comprises, or consists of a polypeptide sequence having a sequence identity of ≥85%, particularly ≥90%, more particularly ≥95% compared to an amino acid sequence designated SEQ ID NO 001, and having at least 90% of the biological function of a sialidase with the sequence SEQ ID NO 001.

### Biological Activity of Sialidase According to the Invention

The biological activity of an AAV-Sia according to the invention may be measured in an in vitro or in vivo system designed to determine the desialylation of AAV-Sia transduced tumour cells, Equivalent biological activity may be defined by induction of at least a 1.2 fold increase in PNA binding, and/or a decrease of MAA II and SNA binding to the surface of a transduced tumour cell compared to a vector lacking the sialidase transgene. Alternatively, a reduction in tumour development, growth rate, or spread in an animal model as demonstrated in FIG. 2 and FIG. 3 of the examples may also be a measure of biological activity of an AAV-Sia.

In the absence of further instructions, to assess whether a variant sialidase has equivalent biological activity, i.e. 90% of the biology activity of a sialidase described herein, biological activity is determined in the following assay. A positive control recombinant AAV2 vector is generated comprising a transgene consisting of a nucleic acid encoding a sialidase polypeptide sequence provided herein to which the variant will be compared, for example SEQ ID NO 011, encoding influenza Neuraminidase 1 protein of SEQ ID NO 001, under control of the CMV promotor as shown in FIG. 1. A test recombinant AAV2 vector is generated by replacing the nucleic acid encoding SEQ ID NO 001, with a nucleic acid encoding the variant sialidase polypeptide to be tested. HEK293 cells are co-transfected with the AAV plasmid bearing each Gene-of-Interest (GOI), together with replication helper-plasmid DNA, encoding the REP and CAP genes of wild type AAV2. Two days after transfection, cell pellets are harvested, and viruses released through 3 freeze/thaw cycles. Viruses are purified by CsCl-gradient ultra-centrifugation, followed by desalting. Viral titre (GC/ml-genome copies/ml) are then determined by means of real-time PCR measurement of the gene of interest. A purity of 90% AAV proteins is confirmed using SDS-gel silver staining, prior to use of viral stocks in experiments. MC38, B16D5, Hela or EMT6 cells (preferably the latter) are plated at 10⁴ cells/well in a 96 well plate, in 0.1 mL of DMEM (Dulbecco's modified Eagle medium, Sigma-Aldrich), 25 mM glucose, supplemented with 1% glutamine, 1% pyruvate, 1% non-essential amino acids, streptomycin penicillin (5000 U/ml) and 10% foetal bovine serum (Sigma-Aldrich) at 37° C. and 5% CO₂. Each viral stock stored in PBS 5% Glycerol is diluted in cell culture medium and applied at a multiplicity of infection (MOI) of 2×10⁵ to three wells of the plate. After 72 hours, the cell media is removed, and the adherent cells are collected in trypsin and washed. The cell pellet of each well is resuspended in 10 μg/ml PNA (Peanut Agglutinin, Vector Biolabs), for 30 minutes. After 2 washes in PBS, the cell pellets are incubated with streptavidin-PE for 30 min (1:500, BD Biosciences). The cells are washed twice in PBS and fixed with IC fixation buffer (ThermoFisher). Flow cytometry is performed to assess the mean fluorescence intensity (MFI) of PE on cells from each well. The average PNA-PE of the replicate wells comprising cells transduced with the test recombinant AAV2 vector, is at least 90% of the average MFI of duplicate samples of the positive control recombinant AAV2 vector encoding a neuraminidase polypeptide sequence defined herein, particularly an N1 polypeptide of sequence SEQ ID NO 001, confirming the sample has 90% of the biological activity of the positive control AAV-Sia.

### Sialidase Viral Vectors According to the Invention

Another aspect of the invention relates to the AAV vector. In certain embodiments, the AAV is a recombinant AAV1. In particular embodiments, the AAV is a recombinant AAV2 vector. AAV2 is the most commonly utilized AAV vector for transgene delivery with broad cellular tropism. However, different engineered, or naturally occurring serotypes with specific tissue tropism may be selected to match the desired tumour cell target. For example, an AAV6 may be selected for targeting a tumour derived from airway epithelial cells, or an AAV8 vector may be chosen to target a hepatic cancer.

In certain embodiments, the AAV-Sia is for use to treat liver cancer, and the AAV-Sia is a AAV8 vector which can induce expression of an influenza neuraminidase polypeptide.

In certain embodiments, the AAV-Sia is for use to treat lung cancer, and the AAV-Sia is a AAV6 vector which can induce expression of an influenza neuraminidase polypeptide.

In certain embodiments, the vector is a recombinant AAV2 vector, which was demonstrated to be effective for inducing functional transgene expression in a range of cancer cell lines in vitro, and has anti-tumour effects in murine models of cancer derived from different tissues (FIGS. 2 and 3).

In certain embodiments the AAV2 vector bears a transgene which encodes an influenza Type A neuraminidase N1 polypeptide. In other embodiments, the AAV-Sia is an AAV2 vector carrying a N2 transgene. In other embodiments, the AAV-Sia is an AAV2 vector carrying a N3 transgene. In other embodiments, the AAV-Sia is an AAV2 vector carrying a N4 transgene. In other embodiments, the AAV-Sia is an AAV2 vector carrying a N5 transgene. In other embodiments, the AAV-Sia is an AAV2 vector carrying a N6 transgene. In other embodiments, the AAV-Sia is an AAV2 vector carrying a N7 transgene. In other embodiments, the AAV-Sia is an AAV2 vector carrying a N8 transgene. In other embodiments, the AAV-Sia is an AAV2 vector carrying a N9 transgene. In other embodiments, the AAV-Sia is an AAV2 vector carrying a N10 transgene. In other embodiments, the AAV-Sia is an AAV2 vector carrying a N11 transgene. In alternative embodiments, the AAV-Sia is an AAV2 vector bearing a transgene encoding a Type B Victoria lineage neuraminidase. In yet another embodiment, the AAV-Sia is an AAV2 vector which encodes a Type B Yamagata lineage neuraminidase.

In other embodiments the AAV-Sia is an AAV2 bearing a transgene which encodes a polypeptide sequence comprising SEQ ID NO 001. In still other embodiments, the AAV-Sia is an AAV2 bearing a transgene which consists of a nucleic acid sequence encoding the protein designated SEQ ID NO 001. In alternative embodiments, the AAV-Sia according to the invention is an AAV2 vector comprising a transgene comprising, or consisting of a nucleic acid sequence encoding a protein selected from those designated SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010. In particular embodiments, the AAV-Sia is an AAV2 vector which encodes an influenza Type A neuraminidase with the sequence SEQ ID NO 001.

Alternative embodiments of the AAV-Sia according to the invention provide an AAV2 vector comprising a sialidase transgene encoding a variant sialidase polypeptide which comprises, or consists of a polypeptide sequence having a sequence identity of ≥85%, particularly ≥90%, more particularly ≥95% compared to a sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010. In particular embodiments, the variant sialidase polypeptide according to this aspect of the invention has at least 90% of the biological activity of the N1 influenza neuraminidase, of sequence SEQ ID NO 001.

In other embodiments, the AAV-Sia is an AAV2 with a transgene comprising a nucleic acid SEQ ID NO 011. In alternative embodiments the AAV-Sia is an AAV2 comprising a sialidase transgene consisting of the sequence SEQ ID NO 012. In alternative embodiments the AAV-Sia is an AAV2 comprising a sialidase transgene consisting of the sequence SEQ ID NO 013. In alternative embodiments the AAV-Sia is an AAV2 comprising a sialidase transgene consisting of the sequence SEQ ID NO 014. In alternative embodiments the AAV-Sia is an AAV2 comprising a sialidase transgene consisting of the sequence SEQ ID NO 015. In alternative embodiments the AAV-Sia is an AAV2 comprising a sialidase transgene consisting of the sequence SEQ ID NO 016. In alternative embodiments the AAV-Sia is an AAV2 comprising a sialidase transgene consisting of the sequence SEQ ID NO 017. In alternative embodiments the AAV-Sia is an AAV2 comprising a sialidase transgene consisting of the sequence SEQ ID NO 018. In alternative embodiments the AAV-Sia is an AAV2 comprising a sialidase transgene consisting of the sequence SEQ ID NO 019. In alternative embodiments the AAV-Sia is an AAV2 comprising a sialidase transgene consisting of the sequence SEQ ID NO 020.

In alternative embodiments, the AAV-Sia is an AAV2 with a transgene has a sequence having a sequence identity of ≥85%, particularly ≥90%, more particularly ≥95% compared to SEQ ID NO 011, SEQ ID NO 012, SEQ ID NO 013, SEQ ID NO 014, SEQ ID NO 015, SEQ ID NO 016, SEQ ID NO 017, SEQ ID NO 018, SEQ ID NO 019, or SEQ ID NO 020, provided the sialidase activity of the polypeptide encoded by the transgene has at least 90% of the biological activity of SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010.

In particular embodiments, the AAV-Sia is an AAV2 carrying a transgene which comprises the nucleic acid sequence SEQ ID NO 011. In more particular embodiments the AAV-Sia is an AAV2 comprising a neuraminidase transgene consisting of the sequence SEQ ID NO 011.

AAV can encapsidate either the plus or minus strand of sialidase sequences into its single-strand DNA genome to generate virions, thus the complimentary nucleic acid sequences of those specified according to this aspect of the AAV-Sia are also encompassed by the invention.

In certain embodiments, the sialidase transgene is located in 3′-direction of, and under control of a promoter sequence operable in a mammalian cell. In certain embodiments, the promoter sequence is operable in a human cell, particularly in a malignant cancer cell. In certain embodiments, the promoter is a ubiquitous promoter. In certain embodiments, the promoter is a cell-specific promoter. In certain embodiments, the promoter is a CMV immediate early promoter. In certain embodiments, the AAV-Sia comprises a nucleic acid sequence encoding an influenza Type A or Type B neuraminidase under control of a CMV immediate early promoter with the nucleic acid sequence SEQ ID NO 021.

In alternative embodiments, the AAV-Sia comprises a nucleic acid sequence encoding an influenza Type A or Type B neuraminidase under control of a human Ef1a promoter, as Ef1a has similar expression properties the CMV promoter tested in the examples, and is often incorporated into commercially available replication deficient-AAV constructs.

A next aspect of the invention relates to an AAV vector comprising a transgene encoding an influenza-derived Type A or Type B neuraminidase, for use in medicine. In particular embodiments, the AAV-Sia is as a medicament for use treating a patient diagnosed with cancer. In more particular embodiments an AAV2 comprising a transgene encoding an influenza N1 neuraminidase polypeptide according to the aspects specified above,

Generally speaking, cancers are classified by the type of tissue they originate from. Epithelial cell-derived cancer, also known as carcinoma, accounts for up to 90% of cancer cases. However, the immune responses required to combat tumours derived from other types of solid tissue (also known as sarcoma) or tumours of mixed origin, are similar. In one embodiment of this aspect of the invention, an AAV-Sia as specified above is provided to treat a form of solid cancer, for example a carcinoma, such as a colon cancer, or a sarcoma, such as a melanoma.

Particular embodiments relate to the use of an AAV-Sia to treat a solid cancer selected from colon cancer, lung cancer, breast cancer, or melanoma.

In certain embodiments, an AAV-Sia is provided for use in a type of cancer for which intratumoural injections of a virus bearing a recombinant transgene is an approved treatment protocols, including, for example, head and neck tumours, triple-negative breast cancer, or cutaneous lymphomas.

In particular embodiments, an AAV-Sia for use in treating cancer according to the invention is used to treat a patient diagnosed with colon cancer, as modelled by the MC38 system.

In particular embodiments, an AAV-Sia for use in treating cancer according to the invention is used to treat a patient diagnosed with lung cancer, shown to be effectively desialylated in Example 7.

In particular embodiments, an AAV-Sia for use in treating cancer according to the invention is used to treat a patient diagnosed with breast cancer, as modelled by the EMT6 cell transduced in the FIG. 2. In particular embodiments, an AAV-Sia for use in treating cancer according to the invention is used to treat a patient diagnosed with triple-negative breast cancer.

In particular embodiments, an AAV-Sia for use in treating cancer according to the invention is used to treat a patient diagnosed with melanoma, as modelled by the B16D5 cells and in vivo tumour model in FIGS. 2 and 3.

In particular embodiments, an AAV-Sia for use in treating cancer according to the invention is used to treat a patient diagnosed with a cutaneous lymphoma.

In some embodiments, In particular embodiments, an AAV-Sia for use in treating cancer according to the invention is used to treat a patient diagnosed with a head and neck cancer.

In some embodiments, the AAV-Sia is for use in treating a patient diagnosed with an epithelial-cell derived tumour. In some embodiments, the cancer is pancreatic cancer. In alternative embodiments, the cancer is prostate cancer.

In some embodiments, the AAV-Sia is for use a patient diagnosed with a cancer characterised by a metastasis to a second location apart from the primary tumour.

Particular embodiments relate to the use of an AAV-Sia according to the invention administered directly into a tumour for example by infusion, by insertion of a micropump, or during a surgical procedure. In particular embodiments, the AAV-Sia is administered to a cancer patient by intratumoural injection. The AAV-Sia according to this aspect of invention may thus plausibly be expected in inhibiting the growth of any type of solid tissue derived cancer or malignant neoplasm, but not for example, a diffuse, blood cell derived cancer.

The broadly beneficial effect of the immunomodulatory actions of a sialidase delivered by means of an AAV vector can be observed in the efficacy when administered alone, or in combination with a form of checkpoint inhibition demonstrated in the examples (FIG. 3).

Other embodiments of the invention relates to use of an AAV-Sia to treat cancer in a patient who has not been administered a checkpoint inhibitor inside a medically relevant window of AAV-Sia use. In other words, an AAV-Sia is given to a patient without combination with an immunotherapeutic agent such as a checkpoint inhibitor antibody such as anti-PD-1 or anti-PD-L1.

In certain embodiments of the AAV-Sia for use to treat a patient diagnosed with cancer, the AAV-Sia is for use in patient who has not been administered a chimeric antigen receptor (CAR) T cell treatment. In other words, an AAV-Sia is given to a patient without combination with an adoptive transfer or recombinant T cells specific for a tumour antigen. In some embodiments, the AAV-Sia is for use in a patient who has not received CAR T cell treatment in the three months prior to AAV-Sia administration. In some embodiments, the AAV-Sia is for use in a patient who is not currently receiving CAR T cell treatment at the time of AAV-Sia administration. In some embodiments, the AAV-Sia is for use in a patient who is scheduled to receive a CAR T cell treatment within three months of AAV-Sia administration.

In certain embodiments of the AAV-Sia for use according to the invention as specified in any of the aspects or embodiments described herein, the AAV-Sia is administered directly to the patient, without being administered to a transgenic cell used in treatment of the patient, such as a CAR-T cell. In particular embodiments, the AAV-Sia may be administered to the patient after or concomitant to cell therapy. In certain more particular embodiments, the AAV-Sia is administered to a patient not having received cell therapy. The term cell therapy specifically relates to administration of cells meant to target the tumour, to the patient.

### Combination Treatments

An alternative embodiment relates to using an AAV-Sia according to the previously specified aspects of the invention to treat a cancer patient in combination with an immunotherapy, particularly selected from an anti-PD-1, anti-PD-L1, or an anti-CTLA-4 checkpoint inhibitor antibody.

In some embodiments the AAV-Sia and the checkpoint inhibitor are delivered together, as a combination medicament administered directly into a solid tumour.

Other embodiments provide an AAV-Sia for intra-tumoural delivery together with parenteral administration of a checkpoint immunotherapy agent. This encompasses the use of an AAV-Sia to treat a patient who has recently received, is currently receiving, or is scheduled to receive a checkpoint inhibitory agent within a medically relevant window of AAV-Sia administration, for example, within 2 months of AAV-Sia administration according to the invention.

Another aspect of the invention relates to a checkpoint inhibitor agent for use in treating cancer, wherein the checkpoint inhibitor is provided for use together with administration of an AAV-Sia according to any one of the aspects of the invention provided above. In some embodiments, the patient is simultaneously administered both agents. In alternative embodiments, both are administered within a medically relevant window, for example in alternating weeks.

In particular embodiments, the immune checkpoint inhibitor agent is an inhibitor of interaction of programmed cell death protein 1 (PD-1) with its receptor PD-L1. In certain embodiments, the immune checkpoint inhibitor agent is selected from the clinically available antibody drugs nivolumab (Bristol-Myers Squibb; CAS No 946414-94-4), pembrolizumab (Merck Inc.; CAS No. 1374853-91-4), pidilizumab (CAS No. 1036730-42-3), atezolizumab (Roche AG; CAS No. 1380723-44-3), and Avelumab (Merck KGaA; CAS No. 1537032-82-8). In certain embodiments, the immune checkpoint inhibitor agent is ipilimumab (Yervoy; CAS No. 477202-00-9).

In particular embodiments, the checkpoint inhibitor agent is a non-agonist ligand for PD-1. In more particular embodiments, the checkpoint inhibitor agent is a non-agonist antibody specific for PD-1.

### Medical Treatment, and Dosage Forms

Similarly, within the scope of the present invention is a method or treating cancer in a patient in need thereof, comprising administering to the patient an AAV-Sia, optionally in addition to a checkpoint inhibitor agent according to the above description.

The term intratumoural administration according to the current specification refers to providing the AAV-sialidase by direct administration into a solid tumour, or into the close vicinity of a tumour, or into the lymph node associated with a tumour. This may be achieved either by a single injection, or intermittent or continuous infusion. In alternative embodiments, intratumoural administration takes place in the context of a surgical intervention, such as direct administration of the AAV-Sia in solution to a biopsy, or tumour resection site.

In certain embodiments, the checkpoint inhibitor agent is an antibody, antibody fragment, an antibody-like molecule or a protein A domains derived polypeptide. In some embodiments, the checkpoint inhibitor agent is an immunoglobulin consisting of two heavy chains and two light chains. In some embodiments, the checkpoint inhibitor agent is a single domain antibody, consisting of an isolated variable domain from a heavy or light chain. In some embodiments, the checkpoint inhibitor agent is a heavy-chain antibody consisting of only heavy chains such as antibodies found in camelids.

In certain embodiments, the checkpoint inhibitor agent is a is an antibody fragment. In certain embodiments, the checkpoint inhibitor agent is a Fab fragment, i.e., the antigen-binding fragment of an antibody, or a single-chain variable fragment, i.e., a fusion protein of the variable region of heavy and the light chain of an antibody connected by a peptide linker.

Similarly, a dosage form for the treatment, or prevention of recurrence of cancer is provided, comprising a non-agonist ligand for a checkpoint inhibitor molecule according to any of the above aspects or embodiments of the invention.

Dosage forms for parenteral administration of a checkpoint inhibitor agent according to the invention may be used, such as subcutaneous, intravenous, intrahepatic or intramuscular injection forms. Optionally, a pharmaceutically acceptable carrier and/or excipient may be present.

Topical administration either the AAV-Sia, or the immune checkpoint inhibitor of the invention is also within the scope of the intratumoural uses of the invention relevant for application to directly to forms of skin cancer, or cutaneous lymphoma. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835).

### Pharmaceutical Compositions and Administration

Another aspect of the invention relates to a pharmaceutical composition comprising the AAV-Sia according to any one of the aspects of the invention related herein. In particular embodiments, the composition is formulated for local administration to a tumour, or the immediate surrounds of a tumour, or a tumour-draining lymph node. In particular embodiments, the composition comprising an AAV-Sia is formulated for intratumoural injection. In other embodiments, the composition comprises both an AAV-Sia together with an immune checkpoint inhibitor of the present invention, and a pharmaceutically acceptable carrier. In further embodiments, the composition comprises at least two pharmaceutically acceptable carriers, such as those described herein.

In certain embodiments of the invention either the AAV-Sia, or the immune checkpoint inhibitor of the invention of the present invention, is typically formulated into pharmaceutical dosage forms to provide an easily controllable dosage of the drug and to give the patient an elegant and easily handled product.

In embodiments of the invention relating to topical uses of the either the AAV-Sia, or the immune checkpoint inhibitor of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition comprising the immune checkpoint inhibitor can be formulated for parenteral administration, for example by intravenous (i.v.) injection or infusion, Intraperitoneal (i.p.), intradermal, subcutaneous or intramuscular administration.

The dosage regimen for the immune checkpoint inhibitor of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of the immune checkpoint inhibitor for a subject of about 50-70 kg. Dosage for the number of AAV-Sia viral particles may be extrapolated from the animal models utilised in the examples, or by similar therapeutic AAV vectors known in the art. The therapeutically effective dosage of a virus, the pharmaceutical composition, or the combinations thereof, is dependent on the body weight, age and individual condition, the disorder or disease or the severity thereof being treated. A physician, or clinician of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of an AAV-Sia according the aspects of the invention identified herein, and optionally an additional immune checkpoint inhibitor ligand, for use in a method of manufacture of a medicament for the treatment or prevention of recurrence of a solid tumour.

Similarly, the invention encompasses methods of treatment of a patient having been diagnosed with a disease associated with a tissue-derived solid tumour. This method entails administering to the patient an effective amount of AAV-Sia as identified herein, particularly by intratumoural administration, and optionally concurrent with, shortly after receiving, or shortly before receiving within a medically relevant window, an immune checkpoint inhibitor antibody as specified in detail herein by a parenteral administration.

Wherever alternatives for single separable features such as, for example, a viral strain, a sialidase sequence, a checkpoint inhibitor agent, or a medical indication are laid out herein as “embodiments”, it is to be understood that such alternatives may be combined freely to form discrete embodiments of the invention disclosed herein. Thus, any of the alternative embodiments for the viral vector may be combined with any of the alternative embodiments of the checkpoint inhibitor, and these combinations may be combined with any medical indication mentioned herein.

The invention further relates to the following items:
A. An adeno-associated virus comprising a transgene encoding a sialidase (AAV-Sia) derived from the influenza virus.
B. An AAV-Sia according to item A, wherein the sialidase is a neuraminidase selected from:
   a. an influenza virus Type A-derived neuraminidase, particularly an N1, N2, N3, N4, N5, N6, N7, N8, N9, N10, or N11 neuraminidase; or
   b. an Influenza Type B Victoria or Yamagata lineage-derived neuraminidase; particularly wherein the sialidase is an N1 neuraminidase.
C. An AAV-Sia according to item A or B, wherein the sialidase comprises, or consists of, a sialidase polypeptide sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010;
   or the sialidase comprises, or consists of, a polypeptide sequence having an identity of ≥85%, particularly ≥90%, more particularly ≥95% compared to said sialidase polypeptide sequence, wherein the polypeptide sequence has at least 90% of the biological activity of said sialidase polypeptide sequence; particularly wherein the sialidase comprises or consists of the polypeptide sequence SEQ ID NO 001.
D. An AAV-Sia according to any one of items A to C, wherein the transgene encoding the sialidase comprises, or consists of a nucleic acid sequence encoding a sialidase polypeptide sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010;
   or a polypeptide sequence having an identity of ≥85%, particularly ≥90%, more particularly ≥95% compared to said sialidase polypeptide sequence, wherein the polypeptide sequence has at least 90% of the biological activity of said sialidase polypeptide sequence;
      particularly wherein the transgene comprises, or consists of a nucleic acid sequence selected from SEQ ID NO 011, SEQ ID NO 012, SEQ ID NO 013, SEQ ID NO 014, SEQ ID NO 015, SEQ ID NO 016, SEQ ID NO 017, SEQ ID NO 018, SEQ ID NO 019, SEQ ID NO 020;
         more particularly wherein the comprises, or consists of the nucleic acid sequence SEQ ID NO 011.
E. An AAV-Sia according to any one of the items A to D, wherein the transgene is comprised within a viral expression element comprising the transgene operably linked to a promotor sequence conferring transgene expression in mammalian cells, particularly wherein the promoter sequence comprises or consists of the cytomegalovirus promoter, more particularly wherein the promoter sequence comprises or consists of the nucleic acid sequence SEQ ID NO 021.
F. An AAV-Sia according to any one of items A to E, wherein the recombinant adeno-associated virus is a replication deficient recombinant adeno-associated virus, particularly a replication deficient adeno-associated Type 2 virus.
G. An AAV-Sia according to any one of items A to F, for use for the treatment, or for prevention of recurrence, of cancer in a patient in need thereof.
H. An AAV-Sia according to item G, wherein the cancer is a solid cancer, particularly a solid cancer selected from colon, lung, breast cancer, and melanoma.
I. An AAV-Sia according to items G or H, wherein the AAV-Sia is administered directly into a tumour, particularly wherein the AAV-Sia is administered by intratumoural injection.
J. An AAV-Sia according to items G to I, wherein the patient is scheduled to receive, is currently receiving, or has recently been administered a checkpoint inhibitor agent.
K. An AAV-Sia according to item J, wherein the checkpoint inhibitor agent is an antibody with specificity for a checkpoint molecule selected from PD-1 or PD-L1, more particularly targeting PD-1.
L. An AAV-Sia according to item J or K, wherein the checkpoint inhibitor agent is administered parenterally.
M. A pharmaceutical composition for use for the treatment, or for prevention of recurrence, of cancer in a patient in need thereof, comprising:
   a. AAV-Sia according to any one of the items A to I, and optionally
   b. a checkpoint inhibitor agent, particularly a PD-1 or PD-L1 ligand, more particularly a PD-1 ligand.
N. A method of treating cancer in a subject in need thereof, the method comprising administering by intratumoural injection to the subject of an effective amount of the AAV-Sia according to any one of the items A to L, or a pharmaceutical composition according to item M.

The invention is further illustrated by the following examples and figures, from which further embodiments and advantages can be drawn. These examples are meant to illustrate the invention but not to limit its scope.

### DESCRIPTION OF THE FIGURES

Table. 1 lists representative sialidase protein sequences isolated from human, avian, and equine hosts, and representative genomic RNA sialidase sequences.

FIG. 1 shows design of AAV2-Sia construct. The AAV2 virus were produced by co-transfecting HEK293 cells with AAV plasmid containing a neuraminidase sequence with helper-plasmids. The neuraminidase (sialidase) was obtained from influenza A H1N1 virus, with a constitutive CMV promoter. In the AAV plasmid, the REP and CAP genes of wild type AAV were deleted, 2 copies of inverted terminal repeats (ITR) remain.

FIG. 2 shows AAV-Sia tumour cell desialylation in vitro. Tumour cell lines A B16D5, B MC38, C EMT6, and D Hela were seeded at 10⁴ cells/well in a 96 well plate (n−1). The AAV-Sia was added at the indicated concentration/cell (multiplicity of infection, MOI), after 72 hours the cells were detached with trypsin and stained with PNA (Peanut Agglutinin) that binds to desialylated glycans or MAA II (Maackia amurensis) and SNA that bind to sialic acid residues in the cell surface. PNA, MAAll or SNA (Sucumbus Nigra) mean fluorescence intensity (MFI) was acquired using flow cytometry.

FIG. 3 shows AAV2-Sia inhibits tumour growth and enhances checkpoint inhibitor treatment. MC38 or B16D5 cells were injected subcutaneously into the flanks of C57Bl6 mice. Tumour growth was measured 3 times weekly until reaching an ethical endpoint tumour volume of ˜1500 mm³. AAV2-Sia or control AAV2-null treatment started when tumours reached ˜50 mm³, four doses (1010 GC) were administered each 2-3 days, injected intratumourally (i.t.) diluted in 50 μl of PBS. a-PD1 antibody was also used as a combination treatment in four doses (10 mg/kg, i.p.). The a-PD1 treatment started with the second dose of the AAV, every 3-4 days. A Tumour growth and B survival in the MC38 colon cancer mouse model: AAV2-sia (n 6), AAV2-null (n-5), AAV2-Sia+a-PD1 (n-5), AAV2-null+a-PD1 (n-6), a-PD1 (n-5) and no treatment (n-5). C Tumour growth and D survival in the aPD-1 resistant B16D5 melanoma mouse model: AAV2-sia (n-10), AAV2-null (n-11), AAV2-Sia+a-PD1 (n-4), AAV2-null+a-PD1 (n-4) and no treatment (n-9). Results are expressed as mean±SEM. p values were calculated using two-way Anova (Bonferroni Test). For the survival curves, p values were calculated using the Gehan-Breslow-Wilcoxon test. *P<0.05, P <0.01 and ***P<0.001.

FIG. 4 shows AAV-Sia viruses counter growth of non-treated distant tumours. AAV-Sia or a AAV2-null control was injected intratumourally into subcutaneous transplanted MC38 tumours (treated) while the contralateral tumours (distant) were left untreated. AAV2-Sia injection directly into a tumour was confirmed to have the expected anti-tumour effect compared to AAV2-null (vehicle) control animals in both ipsilateral (FIG. 4 A) and contralateral (FIG. 4 B) tumour sites.

FIG. 5 shows specific tumour cell-specific sialidase action upon in vitro AAV-Sia transduction of primary non-small cell lung cancer (NSCLC) tumour samples from 3 patients. Isolated primary tumour cells were transduced over 5 days with 2 different concentrations of the AAV2-Sia virus (10⁴ or 10⁵ virus/cell). Flow cytometry analysis was used to measure PNA levels, showing cancer cells within the CD45 negative compartment (FIG. 5 A) were effectively desialylated upon treatment, while CD45+ cells (immune cells) were not affected (FIG. 5 B).

### EXAMPLES

### Example 1: Methods

### Viral Construct

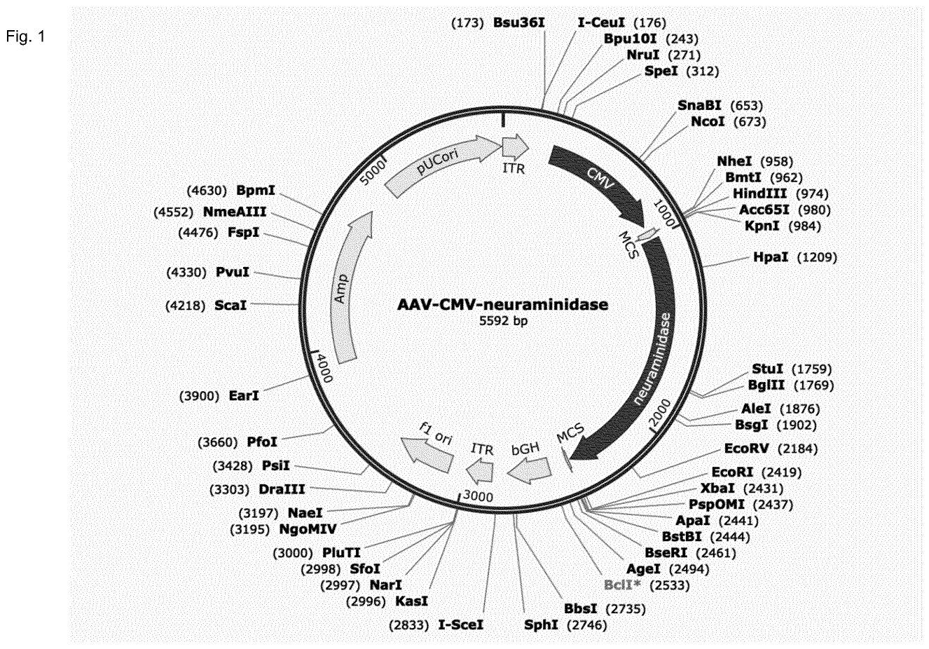

A neuraminidase 1 polypeptide derived from influenza was inserted into a AAV serotype 2 (AAV2) virus, comprising both a capsid and an ITR from AAV2 as shown in FIG. 1. All recombinant AAV viruses and cloning were carried out by Vector BioLabs (lot: 190715 #45, PO: HL_2019_02). In short, HEK293 cells were co-transfected with AAV plasmid bearing a Gene-of-Interest (GOI), together with replication helper-plasmid DNA comprising AAV2 capsid and replication genes. The GOI chosen was Neuraminidase 1 (Influenza A virus H1N1-derived N1, SEQ ID NO 011, RefSeq #NC_026434.1) under control of the CMV (promoter SEQ ID NO 21) (FIG. 1). In the AAV plasmid, the REP and CAP genes of wild type AAV were deleted, leaving 2 copies of ITRs (˜145 bp/each). AAV2-null empty control vectors were prepared in tandem (Vector BioLabs #7026). 2 days after transfection, HEK293 cell pellets were harvested, and viruses released through 3 freeze/thaw cycles. Viruses were purified by CsCl-gradient ultra-centrifugation, followed by desalting. Viral titre (GC/ml-genome copies/ml) was determined through real-time PCR. The purity of AAV proteins were analyzed using SDS-gel silver staining, and only those AAV preparations with >90% purity were used in experiments.

### Viral Stocks

The viral stocks were stored at −80° C., avoiding repeated freeze and thaw cycles. The viral stocks were diluted in PBS 5% Glycerol and diluted in cell culture medium for in vitro experiments or PBS for in vivo treatments.

### Cell Culture

Tumour cell lines were cultured in DMEM (Dulbecco's modified Eagle medium, Sigma-Aldrich), 25 mM glucose, supplemented with 1% glutamine, 1% pyruvate, 1% non-essential amino acids, streptomycin penicillin (5000 U/ml) and 10% foetal bovine serum (Sigma-Aldrich) at 37° C. and 5% CO₂. B16D5, EMT6, MC38 and Hela cells lines were seeded at 10⁴ cells/well in a 6 well plate (n-1). The AAV-Sia was added at different concentration/cell (multiplicity of infection, MOI), and detached after 72 hours the cells with trypsin and stained with PNA (Peanut Agglutinin, Vector Biolabs).

### Flow Cytometry

After AAV2-sia infection, cell lines were detached and stained with biotinylated PNA (10 μg/ml), MAAIl or SNA for 30 min, washed 2 times with PBS and incubated with streptavidin-PE for 30 min (1:500, BD Biosciences). The cells were washed twice and fixed with IC fixation buffer (ThermoFisher). The acquisition was performed using Fortessa LSR II Flow Cytometer (BD Biosciences). The analysis was done using Flowjo and Prism software (Graphpad).

### Animal Experiments

Mouse experiments were approved by the local ethical committee (Kanton Basel Stadt, Switzerland). Males C57Bl/6 mice, 8-10 weeks old were used to perform in vivo experiments. MC38 or B16D5 cells suspension (5×10⁵) in 200 μl of PBS were injected subcutaneously into the flanks of mice. Tumour growth and general health was monitored 3 times per week until tumours reached a maximum size of ˜1500 mm³ (late tumour stage). AAV2-Sia treatment started when tumours reached ˜ 50 mm³, four doses (109 GC) were administered each 3-4 days. Empty vector was used as a control (AAV2-null). All the viruses were injected intratumourally (i.t.), diluted in 50 μl of PBS. In indicated experiments, an anti-PD1 antibody, clone RMP1-14 (BioXcell), was also used as a combination treatment in four doses (10 mg/kg, i.p.). The a-PD1 treatment started with the second dose of the AAV, also every 3-4 days.

### Primary Tumour Cells

Primary samples were obtained from the Division of thoracic surgery of the University hospital authorized be the local ethical committee (EKNZ 2018-01990). For the preparation of single cell suspensions, tumors were collected, surgical specimens were mechanically dissociated and subsequently digested using accutase (PAA Laboratories, Germany), collagenase IV (Worthington, USA), hyaluronidase (Sigma, USA) and DNase type IV (Sigma, USA) for 1 h at 37° C. under constant agitation The digested primary tumours were cultured in 24-well plates overnight at 5×10⁴ cells per well, at the same day samples were transduced with AAV-Sia over 5 days with 2 different concentrations (10⁴ or 10⁵ Virus/cell). Both adherent and non-adherent cells were harvested on day 5, and stained with biotinylated PNA for flow cytometry analysis as above.

### Example 2: Design of an AAV2-NA

An adeno-associated virus (AAV2-Sia) was engineered such that upon transduction of mammalian cells it may produce a sialidase. The influenza neuraminidase N1 gene (SEQ ID NO 011, encoding SEQ ID NO 001) was amplified/synthesized and inserted by ligation into a commercial replication-deficient AAV serotype 2 virus in an expression cassette under control of the constitutive CMV promotor (FIG. 1). A vector lacking the N1 gene insert was used as a control (AAV2-null).

### Example 3: AAV2-NA Removes SA In Vitro

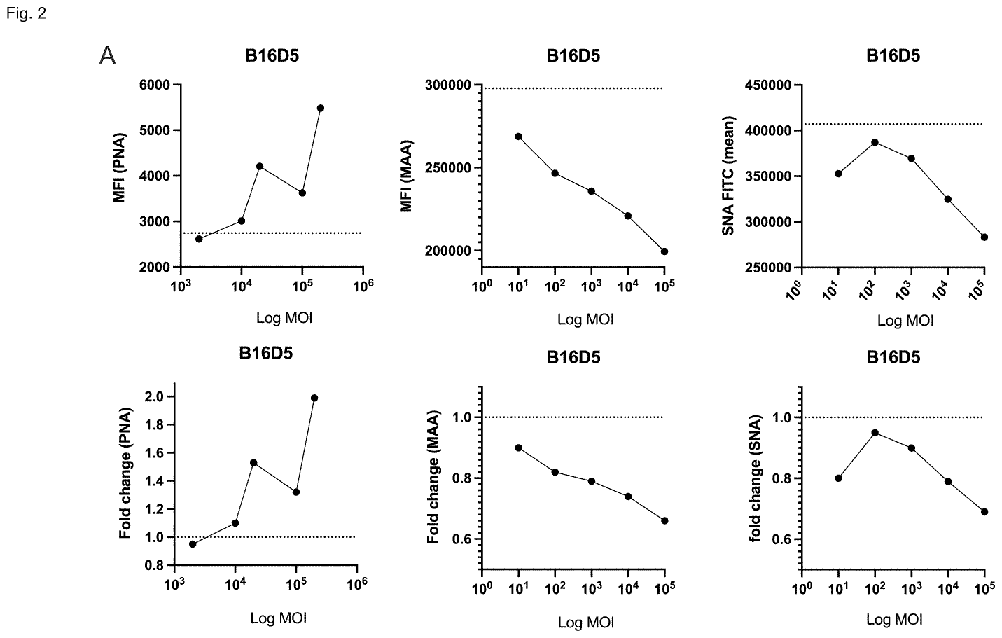

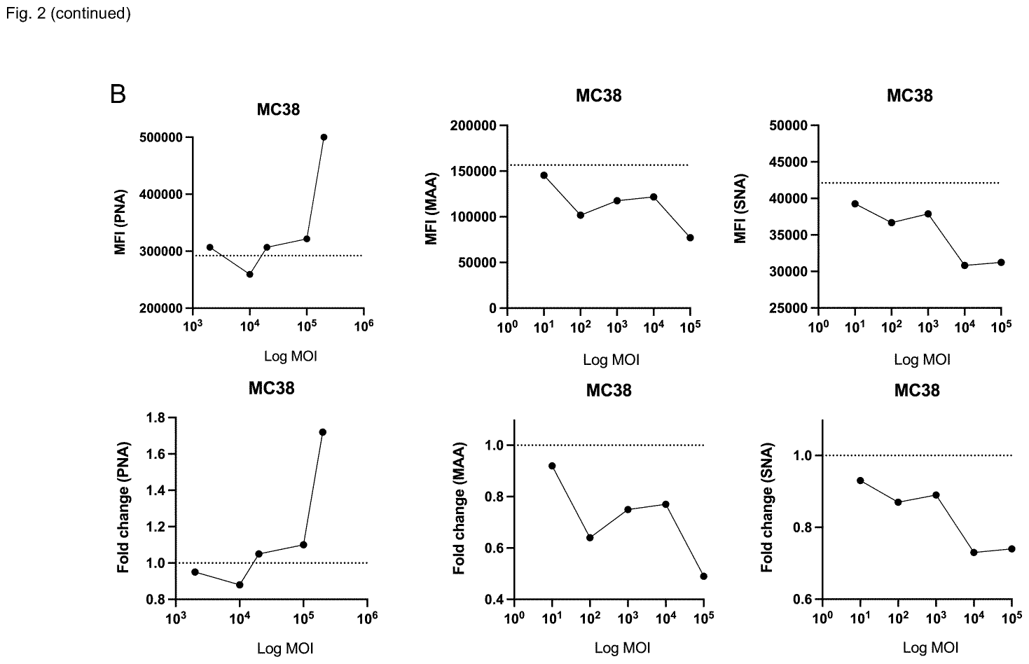

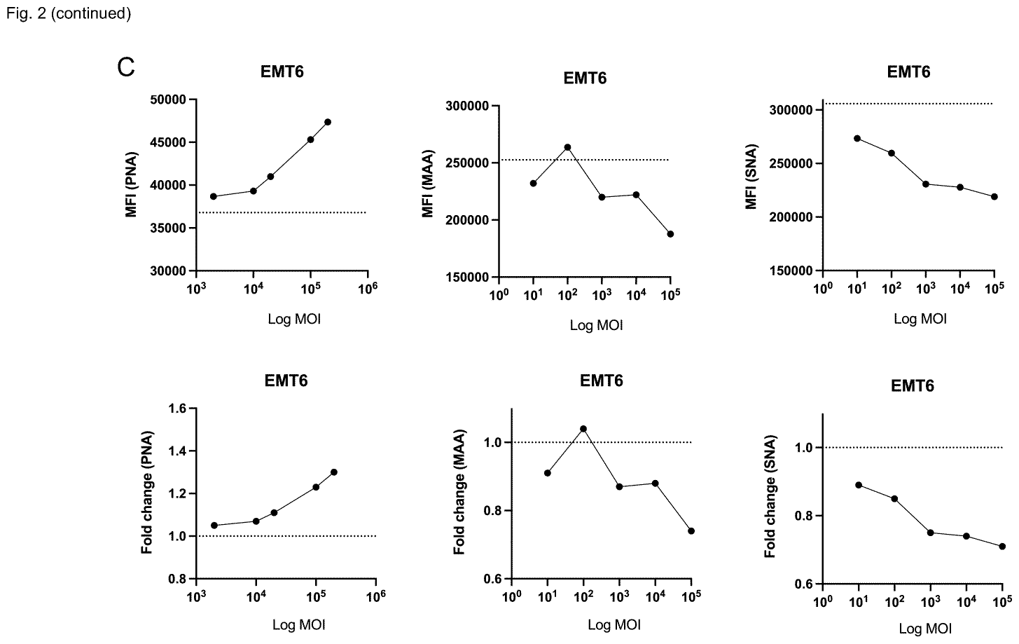

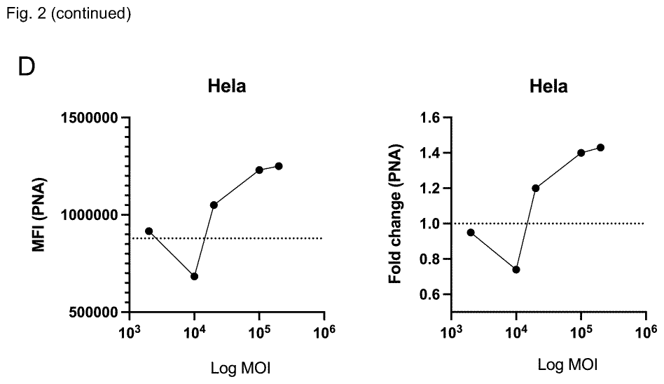

In vitro analyses of tumour cell infection were performed. To determine whether the AAV-Sia induced functional expression of a sialidase in transduced cells, the level of desialylation was analysed by flow cytometry and direct PNA staining, to test the enzymatic activity, i.e. desialylation of in vitro transduced tumour cells. This demonstrated AAV2-Sia induced desialysation of tumour cells upon increasing exposure to AAV2-SIa, as shown by increased binding of the lectin PNA (peanut agglutinin) binding to desialylated glycan residues (FIG. 2). The sialylation was also accessed using the lectins MAA II (Maackia amurensis) and SNA (Sambucus Nigra) that directly binding to sialic acid residues, transduced cells consistently decreased the binding (FIG. 2).

### Example 4: AAV2-Sia Inhibits Tumour Growth in Preclinical Mouse Models

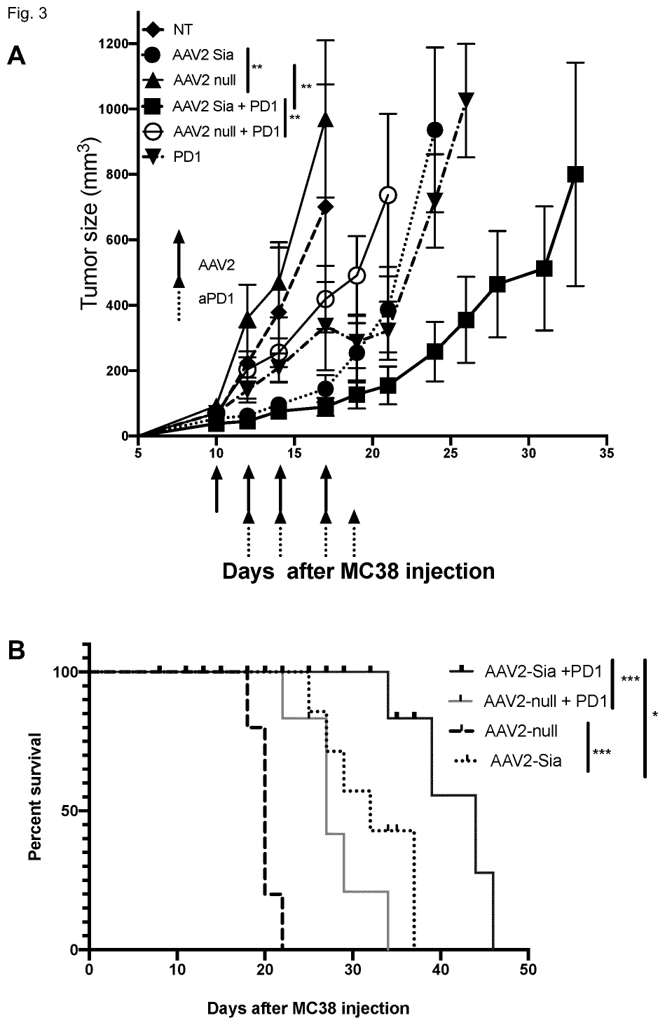

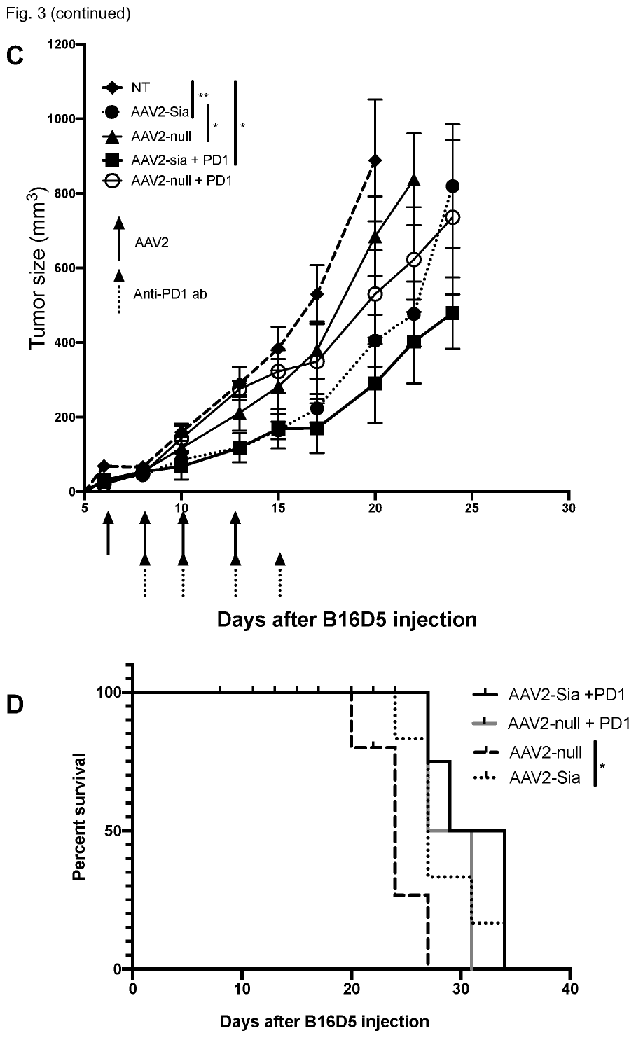

The efficacy of these AAV2-Sia viruses was tested by administering the virus intratumourally into subcutaneous transplanted Bl6D5 melanoma and MC38 colon cancer-derived tumours. The AAV2-Sia virus was applied in a syngeneic tumour model of subcutaneously implanted tumour cells in C57Bl6 mice, where an anti-tumour effect was observed compared the AAV2-Sia and AAV2-null treatment (FIG. 3).

### Example 5: AAV2-Sia has an Additive Benefit to Checkpoint Inhibitor Treatment

Combination therapy of AAV2-sia and a-PD1 antibody was tested by co-administration of a-PD1 antibody after the second dose of the AAV2-Sia, in both the MC38 and B15D5 models. The combination showed additive benefit in the responsive MC38 tumour model, where a reduced rate of tumour growth and increased survival were observed comparing AAV2-Sia versus AAV2-Sia+aPD1, compared to control AAV2-null and AAV2-null+a-PD1 groups (FIG. 3). At day 35, AAV-Sia treated animal showed a distinct 50% survival advantage compared to anti-PD-1 treatment alone. An unexpected synergistic survival advantage was conferred by combined treatment with both an AAV-Sia according to the invention, and non-agonist anti-PD-1 antibody. After day 40 approximately 80% of animals receiving systemic injections of anti-PD1 antibody combined with local AAV-Sia administration were alive, while no animals in groups receiving either treatment alone survived. Melanoma B16 tumours, a cancer model more resistant to immunotherapy, showed no significant decrease in tumour burden upon immune checkpoint blockade when paired with AAV2-null. However, AAV2-Sia drove a small but significant decrease in the rate of tumour growth, which was enhanced with the addition of aPD-1 treatment, suggesting AAV2-Sia induced susceptibility of an immunotherapy-resistant cancer. Combination treatment also conferred a synergistic survival advantage at day 34 following implant of this treatment resistant tumour, with 50% of animals surviving to this timepoint, compared to just 20% in the control group, and none in the anti-PD1 treated group.

### Example 6: AAV2-Sia Limits Abscopal Tumour Growth

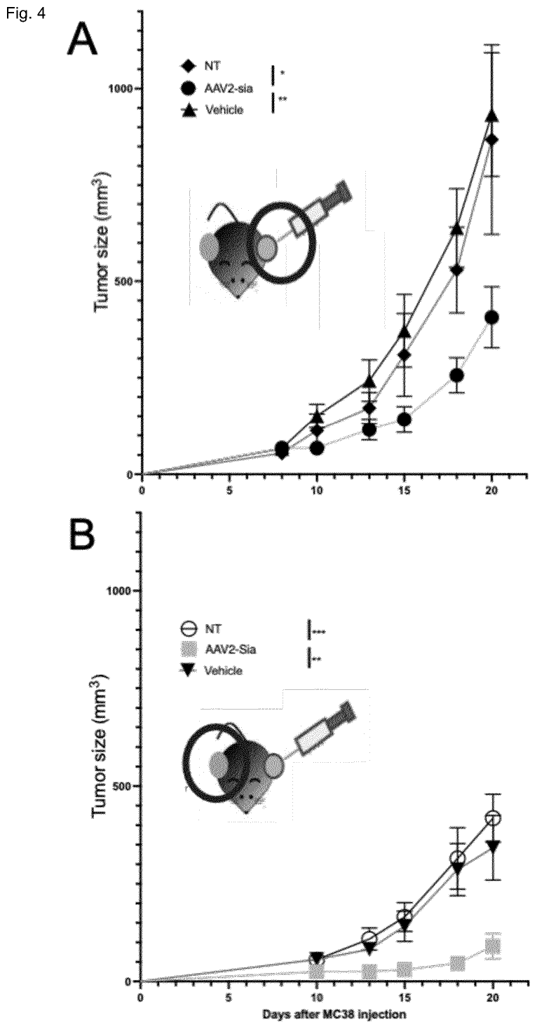

Intratumoural administration of AAV-Sia is demonstrated above to deliver local effects at the tumour where the injection is received. The inventors then asked if the AAV-Sia activates systemic adaptive immunity, with the potential to abrogate growth of secondary, metastatic tumours at distant sites within the host. The efficacy AAV2-Sia viruses in non-treated distant tumours was tested by administering the virus intratumourally into subcutaneous transplanted MC38 tumours (treated) while the contralateral tumours (distant) were left untreated. AAV2-Sia injection directly into a tumour was confirmed to have the expected anti-tumour effect compared to AAV2-null (vehicle) control animals (FIG. 4 A). Importantly, growth of the contralateral tumour that was had not directly received the virus was also inhibited, showing a systemic anti-tumour immune response generated by local AAV-Sia application can counter tumour growth throughout the host to control metastatic cancer (FIG. 4 B).

### Example 7: AAV-Sia Safety Profile

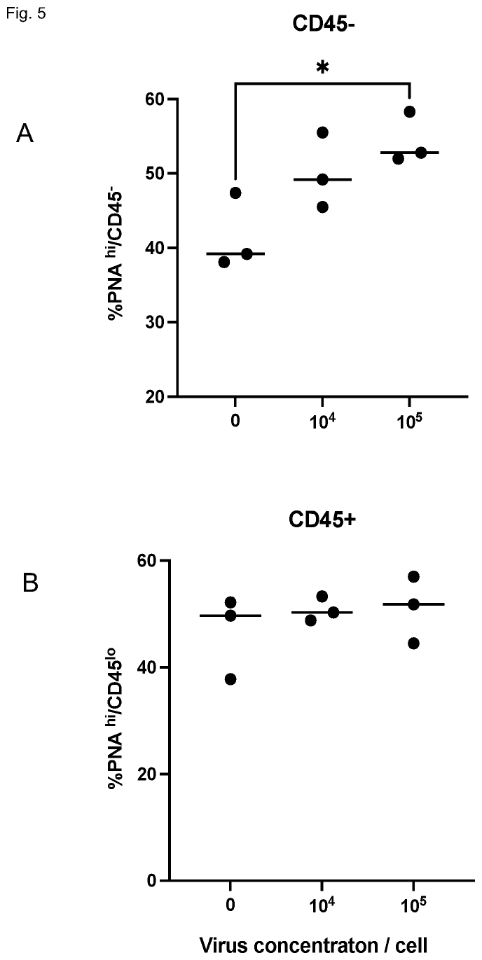

Having demonstrated that immune cells were mediating local and systemic protection against tumours in AAV-Sia treated animals, the investigators considered what effect sialidase expression by virus-infected cells might have on immune cells. T cells are a vital component of anti-tumour immune responses, particularly in patients receiving checkpoint inhibition. Like tumour cells, the membrane lipids of T cell are decorated with glycans comprising terminal sialic acid residues, and such residues are involved in almost every aspect of T cell fate and function, from cell maturation, differentiation, and migration to cell survival and cell death. Ideally, an AAV-Sia composition for use in treating cancer, will induce specific desialylation of cancer cells upon AAV-Sia of primary tumours, with limited enzymatic effect on bystander cells, such as infiltrating immune cells, in order not to perturb protective immune cell migration and acquisition of effector functions. To assess any bystander effects of sialidase expressed within tumours, AAV-Sia was used to infect a mixed cell culture of CD45+ immune cells and CD45-cancer cells obtained from tissue samples derived from digested tumours samples from 3 different patients with lung cancer in vitro, using the lectin PNA. The primary tumour cells were transduced over 5 days with 2 different concentrations of the AAV2-Sia virus (10⁴ or 10⁵ Virus/cell). Flow cytometry analysis of PNA levels following viral transduction demonstrated cancer cells, within the CD45 negative compartment (FIG. 5 A) were effectively desialylated upon treatment, while CD45+ cells (immune cells) were not affected (FIG. 5 B). AAV-Sia thus exhibits a high level of specificity of sialidase action for tumour cells, and has less effect of immune cells within a mixed primary cell population obtained from patient samples. This specific action makes AAV-Sia a desirable alternative to other forms of sialidase administration in the context of cancer, due to a lack of bystander effects on the recruitment and effector functions of local protective immune cell responses.

| | | SEQ | SEQ ID |
|---|---|---|---|
| Neuraminidase | Database | protein | DNA |
| Influenza A H1N1 | Uniprot: C3W6G3 | 001 | 011 |
| Influenza A H3N2 | RefSeq: NC_007368.1 | 002 | 012 |
| Influenza A H7N3 | Uniprot: P03476 | 003 | 013 |
| Influenza A H8N4 | Uniprot: P03477 | 004 | 014 |
| Influenza A H6N5 | Uniprot: P03478 | 005 | 015 |
| Influenza H11N6 | Uniprot: Q6XV27 | 006 | 016 |
| Influenza H7N7 | Uniprot: P88838 | 007 | 017 |
| Influenza H3N8 | Uniprot: Q07599 | 008 | 018 |
| Influenza H11N9 | Uniprot: P03472 | 009 | 019 |
| Influenza B | RefSeq: NC_002209.1 | 010 | 020 |

Table. 1 shows representative sialidase protein sequences isolated from human, avian, and equine hosts, and representative genomic RNA sialidase sequences.

1. An adeno-associated virus comprising a transgene encoding a sialidase (AAV-Sia), wherein the sialidase is derived from the influenza virus, for use for the treatment, or for the prevention of recurrence of cancer in a patient in need thereof.

2. The AAV-Sia for use according to claim 1, wherein the sialidase is a neuraminidase selected from:
a. an influenza virus Type A-derived neuraminidase, particularly an N1, N2, N3, N4, N5, N6, N7, N8, N9, N10, or N11 neuraminidase; or
b. an influenza Type B Victoria, or Yamagata lineage-derived neuraminidase;
particularly wherein the sialidase is an N1 neuraminidase.

3. The AAV-Sia for use according to claim 1, wherein the sialidase comprises, or consists of, a sialidase polypeptide sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010;
or the sialidase comprises, or consists of, a polypeptide sequence having an identity of ≥85%, particularly ≥90%, more particularly ≥95% compared to said sialidase polypeptide sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010, wherein the polypeptide sequence has at least 90% of the biological activity of said sialidase polypeptide sequence;
particularly wherein the sialidase comprises or consists of the polypeptide sequence SEQ ID NO 001, or the sialidase comprises, or consists of, a polypeptide sequence having an identity of ≥85%, particularly ≥90%, more particularly ≥95% compared to SEQ ID NO 001, and has at least 90% of the biological activity of sialidase polypeptide sequence SEQ ID NO 001.

4. The AAV-Sia for use according to claim 1, wherein the transgene encoding the sialidase comprises, or consists of a nucleic acid sequence encoding a sialidase polypeptide sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, or SEQ ID NO 010;
or a polypeptide sequence having an identity of ≥85%, particularly ≥90%, more particularly ≥95% compared to said sialidase polypeptide sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010, wherein the polypeptide sequence has at least 90% of the biological activity of said sialidase polypeptide sequence;
particularly wherein the transgene comprises, or consists of a nucleic acid sequence selected from SEQ ID NO 011, SEQ ID NO 012, SEQ ID NO 013, SEQ ID NO 014, SEQ ID NO 015, SEQ ID NO 016, SEQ ID NO 017, SEQ ID NO 018, SEQ ID NO 019, or SEQ ID NO 020;
more particularly wherein the transgene comprises, or consists of the nucleic acid sequence SEQ ID NO 011.

5. The AAV-Sia for use according to claim 1, wherein the transgene is comprised within a viral expression element comprising the transgene operably linked to a promotor sequence conferring transgene expression in mammalian cells, particularly wherein the promoter sequence comprises or consists of the cytomegalovirus promoter,
more particularly wherein the promoter sequence comprises or consists of the nucleic acid sequence SEQ ID NO 021.

6. The AAV-Sia for use according to claim 1, wherein the adeno-associated virus is a replication deficient recombinant adeno-associated virus.

7. The AAV-Sia for use according to claim 1, wherein the adeno-associated virus is an adeno-associated Type 2 virus.

8. An AAV-Sia for use according to claim 1, wherein the cancer is a solid cancer, particularly a solid cancer selected from liver cancer, prostate cancer, pancreatic cancer, colon cancer, cervical cancer, lung cancer, breast cancer, and melanoma.

9. The AAV-Sia for use according to claim 1, wherein the cancer is characterised as a metastatic cancer.

10. An AAV-Sia for use according to claim 1, wherein the AAV-Sia is administered directly into a tumour, particularly wherein the AAV-Sia is administered by intratumoural injection.

11. An AAV-Sia for use according to claim 1, wherein the patient is scheduled to receive, is currently receiving, or has recently been administered, a checkpoint inhibitor agent.

12. An AAV-Sia for use according to claim 11, wherein the checkpoint inhibitor agent is an antibody with specificity for a checkpoint molecule selected from PD-1 or PD-L1, more particularly an antibody with specificity for PD-1.

13. An AAV-Sia for use according to claim 11, wherein the checkpoint inhibitor agent is administered parenterally.

14. A pharmaceutical composition for use for the treatment of cancer, or for the prevention of recurrence of cancer in a patient in need thereof, comprising an AAV-Sia as specified in claim 1.

15. A pharmaceutical composition for use according to claim 14, formulated for intratumoural administration.


SEQUENCE LISTING (21 sequences)

SEQ ID NO: 1 - protein, 469 aa, Influenza A virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 2 - protein, 469 aa, Influenza A virus
MNPNQKIITIGSVSLTISTICFFMQIAILITTVTLHFKQYEFNSPPNNQVMLCEPTIIERNITEIVYLTNTTIEKEMCPKLAEYRNWSKPQCDITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKKILRTQESECVCINGTCTVVMTDGSASGKADTKILFIEEGKIIHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKEETEVLWTSNSIVVFCGTSGTYGTGSWPDGADINLMPI

SEQ ID NO: 3 - protein, 469 aa, Influenza A virus
MNPNQKIITIGVVNTTLSTIALLIGVGNLVFNTVIHEKIGNHQTVIHPTITTPAVPNCSDTIITYNNTVINNITTTIITEAERLFKPPLPLCPFRGFFPFHKDNAIRLGENKDVIVTREPYVSCDNDNCWSFALAQGALLGTKHSNGTIKDRTPYRSLIRFPIGTAPVLGNYKEICIAWSSSSCFDGKEWMHVCMTGNDNDASAQIIYAGRMTDSIKSWRKDILRTQESECQCIGGTCVVAVTDGPAANSADHRVYWIREGRIVKYENVPKTKIQHLEECSCYVDIDVYCICRDNWKGSNRPWMRINNETILETGYVCSKFHSDTPRPADPSTVSCDSPSNINGGPGVKGFGFKAGNDVWLGRTVSTSGRSGFEIIKVTDGWINSPNHAKSVTQTLVSNNDWSGYSGSFIVKTKGCFQPCFYVELIRGRPNKNDDVSWTSNSIVTFCGLDNEPGSGNWPDGSNIGFMPK

SEQ ID NO: 4 - protein, 470 aa, Influenza A virus
MNPNQKIITIGSASIVLTTIGLLLQITSLCSIWFSHYNQVTQPHEQACSNNTTNYYNETFVNVTNVQNNYTTIIEPSAPNVVHYSSGRDLCPVKGWAPLSKDNGIRIGSRGEVFVIREPFISCSISECRTFFLTQGALLNDKHSNGTVKDRSPFRTLMSCPMGVAPSPSNSRFESVAWSATACSDGPGWLTLGITGPDATAVAVLKYNGIITDTLKSWKGNIMRTQESECVCQDEFCYTLITDGPSNAQAFYKILKIRKGKIVSVKDVNATGFHFEECSCYPSGTDVECVCRDNWRGSNRPWIRFNSDLDYQIGYVCSGIFGDNPRPVDGIGSCNSPVNNGKGRYGVKGFSFRYGDGVWIGRTKSLESRSGFEMVWDANGWVSTDKDSNGVQDIIDNNNWSGYSGSFSIRWETTGRNCTVPCFWVEMIRGQPKEKTIWTSGSSIAFCGVNSDTTGWSWPDGALLPFDIDK

SEQ ID NO: 5 - protein, 468 aa, Influenza A virus
MNPNQKIITIGSASLGLVIFNILLHVASITLGIISVTKDNKVHICNTTEVYNETVRVETVVIPVNNTIYLNHEPEFLNNTEPLCDVSGFAIVSKDNGIRIGSRGHIFVIREPFVSCGPSECRTFFLTQGALLNDKHSNNTVKDRSPYRALMSVPLGSSPNAYQAKFESVGWSATACHDGKKWMAIGVSGADDDAYAVIHYGGVPTDVIRSWRKQILRTQESSCVCIKGECYWVMTDGPANNQASYKIFKSQKGMVVDEKEISFQGGHIEECSCYPNMGKVECVCRDNWNGMNRPILIFDEKLEYEVGYLCAGIPTDTPRVQDSSFTGSCTNAVGRSGTNNYGVKGFGFRQGNSVWAGRTISVSSRSGFEVLLIEDGWIRPSKTISKKVEVLNNKNWSGYSGAFTIPTAMTSKNCIVPCFWLEMIRGKPEERTSIWTSSSSTVFCGVSSEVPGWSWDDGAILPFDIDKM

SEQ ID NO: 6 - protein, 470 aa, Influenza A virus
MNPNQKIICISATGMTLSVVSLLVGIANLGLNIGLHYKVGDTPNVNIPNVNGTNSTTTIINNNTQNNFTNITNIIQSKGGERTFLNLTKPLCEVNSWHILSKDNAIRIGEDAHILVTREPYLSCDPQGCRMFALSQGTTLRGRHANGTIHDRSPFRALISWEMGQAPSPYNTRVECIGWSSTSCHDGMSRMSICMSGPNNNASAVVWYGGRPITEIPSWAGNILRTQESECVCHKGVCPVVMTDGPANNRAATKIIYFKEGKIQKIEELAGNAQHIEECSCYGAGGVIKCICRDNWKGANRPVITIDPEMMTHTSKYLCSKVLTDTSRPNDPTNGNCDAPITGGSPDPGVKGFAFLDGENSWLGRTISKDSRSGYEMLKVPNAETDIQSGPISNQVIVNNQNWSGYSGAFIDYWANKECFNPCFYVELIRGRPKESSVLWTSNSIVALCGSKKRLGSWSWHDGAEIIYFE

SEQ ID NO: 7 - protein, 469 aa, Influenza A virus
MNPNQKLFASSGIAIALGIINLLIGISNMSLNISLYSKGENHKSDNLTCTNINQNNTTMVNTYINNTTIIDKNTKMENPGYLLLNKSLCNVEGWVVIAKDNAIRFGESEQIIVTREPYVSCDPLSCKMYALHQGTTIRNKHSNGTTHDRTAFRGLISTPLGNPPTVSNSEFICVGWSSTSCHDGVSRMTICVQGNNENATATVYYNKRLTTTIKTWAKNILRTQESECVCHNSTCVVVMTDGPANNQAFTKVIYFHKGTIIKEEPLKGSAKHIEECSCYGHNQRVTCVCRDNWQGANRPVIEIDMNNLEHTSRYICTGVLTDTSRPKDKAIGECFNPITGSPGAPGIKGFGFLNENNTWLGRTISPKLRSGFEMLKIPNAGTDPDSKIKERQEIVGNDNWSGYSGSFIDYWNDNSECYNPCFYVELIRGRPEEAKYVEWTSNSLIALCGSPIPVGSGSFPDGAQIKYFS

SEQ ID NO: 8 - protein, 470 aa, Influenza A virus
MNPNQKIITIGSISLGLVVFNVLLHVVSIIVTVLVLGKGGNNGICNETVVREYNETVRIEKVTQWHNTNVVEYVPYWNGGTYMNNTEAICDAKGFAPFSKDNGIRIGSRGHIFVIREPFVSCSPIECRTFFLTQGSLLNDKHSNGTVKDRSPFRTLMSVEVGQSPNVYQARFEAVAWSATACHDGKKWMTVGVTGPDSKAVAVIHYGGVPTDVVNSWAGDILRTQESSCTCIQGDCYWVMTDGPANRQAQYRIYKANQGRIIGQTDISFNGGHIEECSCYPNDGKVECVCRDGWTGTNRPVLVISPDLSYRVGYLCAGIPSDTPRGEDTQFTGSCTSPMGNQGYGVKGFGFRQGTDVWMGRTISRTSRSGFEILRIKNGWTQTSKEQIRKQVVVDNLNWSGYSGSFTLPVELSGKDCLVPCFWVEMIRGKPEEKTIWTSSSSIVMCGVDYEVADWSWHDGAILPFDIDKM

SEQ ID NO: 9 - protein, 470 aa, Influenza A virus
MNPNQKILCTSATALVIGTIAVLIGITNLGLNIGLHLKPSCNCSHSQPEATNASQTIINNYYNDTNITQISNTNIQVEERAIRDFNNLTKGLCTINSWHIYGKDNAVRIGEDSDVLVTREPYVSCDPDECRFYALSQGTTIRGKHSNGTIHDRSQYRALISWPLSSPPTVYNSRVECIGWSSTSCHDGKTRMSICISGPNNNASAVIWYNRRPVTEINTWARNILRTQESECVCHNGVCPVVFTDGSATGPAETRIYYFKEGKILKWEPLAGTAKHIEECSCYGERAEITCTCRDNWQGSNRPVIRIDPVAMTHTSQYICSPVLTDNPRPNDPTVGKCNDPYPGNNNNGVKGFSYLDGVNTWLGRTISIASRSGYEMLKVPNALTDDKSKPTQGQTIVLNTDWSGYSGSFMDYWAEGECYRACFYVELIRGRPKEDKVWWTSNSIVSMCSSTEFLGQWDWPDGAKIEYFL

SEQ ID NO: 10 - protein, 466 aa, Influenza B virus
MLPSTVQTLTLLLTSGGVLLSLYVSASLSYLLYSDVLLKFSSTKTTAPTMSLECTNASNAQTVNHSATKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 11 - DNA, 1410 bp, Influenza A virus
atgaatccaaaccaaaagataataaccattggttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtgtaagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacaatatattgagaacacaagagtctgaatgtgcatgtgtaaatggttcttgctttactgtaatgaccgatggaccaagtaatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaagggttttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaagtaa

SEQ ID NO: 12 - DNA, 1410 bp, Influenza A virus
atgaatccaaatcaaaagataataacgattggctctgtttctctcaccatttccacaatatgcttcttcatgcaaattgccatcctgataaccactgtaacattgcatttcaagcaatatgaattcaactcccccccaaacaaccaagtgatgctgtgtgaaccaacaataatagaaagaaacataacagagatagtgtatctgaccaacaccaccatagagaaggaaatgtgccccaaactagcagaatacagaaattggtcaaagccgcaatgtgacattacaggatttgcacctttttctaaggacaattcgattaggctttccgctggtggggacatctgggtgacaagagaaccttatgtgtcatgcgaccctgacaagtgttaccaatttgcccttggacagggaacaacactaaacaacgtgcattcaaatgacacagtacatgataggaccccttatcggaccctattgatgaatgaattaggtgttccatttcatctggggaccaagcaagtgtgcatagcatggtccagctcaagttgtcacgatggaaaagcatggctgcatgtttgtgtaacgggggatgataaaaatgcaactgctagcttcatttacaatgggaggcttgtagatagtattgtttcatggtccaaaaaaatcctcaggacccaggagtcagaatgcgtttgtatcaatggaacttgtacagtagtaatgactgatgggagtgcttcaggaaaagctgatactaaaatactattcattgaggaggggaaaatcattcatactagcacattgtcaggaagtgctcagcatgtcgaggagtgctcctgctatcctcgatatcctggtgtcagatgtgtctgcagagacaactggaaaggctccaataggcccatcgtagatataaacataaaggattatagcattgtttccagttatgtgtgctcagggcttgttggagacacacccagaaaaaacgacagctccagcagtagccattgcttggatcctaacaatgaagaaggtggtcatggagtgaaaggctgggcctttgatgatggaaatgacgtgtggatgggaagaacgatcagcgagaagttacgctcaggatatgaaaccttcaaagtcattgaaggctggtccaaacctaattccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtgttttgtggcacctcaggtacatatggaacaggctcatggcctgatggggcggacatcaatctcatgcctatataa

SEQ ID NO: 13 - DNA, 1410 bp, Influenza A virus
atgaatccaaatcagaagataataacaatcggggtagtgaacactactctatcaacaatagcccttctcattggagtgggaaatctggttttcaacacagtcatacatgagaaaatagggaatcaccaaacagtgattcacccaacaataacgactcctgcagtaccaaactgcagtgacactataataacatacaacaacactgtgataaacaacataacaacaacaataataactgaagcggaaaggctttttaagcctccactgccgctgtgccccttccgaggattcttcccttttcacaaggacaatgcaatacgattgggtgagaacaaggacgtcatagtcacaagagaaccttatgttagctgcgataatgacaattgttggtccttcgctctcgcacaaggagcactgttagggactaaacatagcaatggaaccatcaaagacagaacaccgtataggtctctaatccgattcccaataggaacagccccagtactgggaaattacaaggagatatgcattgcttggtcgagtagcagttgctttgacgggaaagagtggatgcatgtatgcatgacagggaacgataatgatgcaagtgcccaaataatatatgcagggagaatgacagactccatcaaatcatggagaaaggacatactaaggacccaagagtccgaatgtcaatgcattggcggaacttgtgttgttgctgttacagatggccctgctgctaatagtgcagatcatagggtttattggatacgggaggggagaatagtgaagtatgaaaatgtccctaaaacaaagatacaacacttagaagagtgttcctgctatgtggacatcgatgtgtactgtatatgcagggacaattggaagggttccaacagaccttggatgagaatcaacaacgagaccatactggaaacaggatatgtgtgcagcaaatttcactcagacactcccaggccagctgacccctcaacagtatcatgtgattctccaagcaacattaatggaggacccggagttaagggatttggcttcaaagccggcaatgatgtgtggttgggcaggacagtgtcaactagtggtagatcaggctttgaaatcatcaaagttacagatgggtggatcaactctcccaatcacgccaaatcagttacacaaacactagtgtccaacaatgattggtcaggctattcaggtagtttcattgtcaaaaccaagggctgtttccagccctgcttttatgtcgaacttatacgaggaaggcccaacaagaatgatgatgtctcttggacaagcaatagtatagttactttctgtgggttagacaatgaacctggatcgggaaattggccggatggttccaacattgggtttatgcccaagtaa

SEQ ID NO: 14 - DNA, 1413 bp, Influenza A virus
atgaatccaaatcagaaaatcataaccatcggtagtgcgagtattgtattaacaacaatagggttgctcctccagataacaagtttatgctcaatatggtttagccattacaaccaagtgacccaaccgcatgaacaagcctgttcgaacaacacaacgaattactataatgagacctttgtcaatgtcaccaatgtgcagaacaactataccacaataattgaaccctcagcccccaatgtggttcactactctagtggaagagacttgtgcccagtaaaggggtgggcacctctgagtaaggacaatggaattagaattggatcccgaggcgaagtatttgtcatacgggagcctttcatatcatgttccattagtgaatgcaggacttttttcttaactcagggagccctcctcaatgacaagcactcaaatggaacagtaaaagacaggagtccctttcgtacattaatgagttgtcctatgggagttgccccctctcctagcaacagccgctttgaatctgtggcatggtctgccactgcatgcagtgatggacccggttggttaacattagggatcactggcccagatgctactgctgtggcagtactgaaatacaatggtataataacagacacgttaaaaagctggaagggtaatattatgcgaacacaagagtctgaatgcgtatgccaggatgaattttgttataccttaataacagacggaccgtccaacgcacaagctttctataagatactaaagatcagaaaagggaaaatagtaagtgtgaaggatgtgaatgcaacagggtttcattttgaagaatgttcctgttacccgagcgggacagatgttgaatgtgtctgcagggacaattggcgggggagcaatcgaccgtggataagattcaacagtgatcttgattaccaaattggctatgtatgtagtgggatatttggggacaatcccaggcctgtggatggcataggctcgtgtaacagcccagtcaacaatgggaaaggaagatatggagtgaagggattcagctttagatatggggatggtgtttggatagggaggacaaaaagcttggaatccagaagcggttttgagatggtctgggatgctaatgggtgggtatcgacagacaaggattcaaatggtgtacaggatattatagacaacaacaattggtctggttacagcggaagcttcagcattagatgggagacaacaggcaggaattgcactgtcccatgtttttgggttgagatgataagagggcagcctaaagaaaaaaccatatggactagtggtagtagcattgcattctgtggtgttaattctgataccacaggttggtcatggcccgatggcgctctgttgccctttgacatagacaagtaa

SEQ ID NO: 15 - DNA, 1407 bp, Influenza A virus
atgaatccaaatcagaaaataataacaattggctcagcatcgttgggattagtcattttcaacattcttcttcatgtggcatcaataaccttgggaataatatcagtaaccaaagacaacaaagtacatatctgtaatacgactgaggtgtataatgaaacggtgagagtggaaacagtggtaatccctgtcaacaacaccatatatttgaatcatgagccagaattcctcaacaacacggaacctctctgtgatgtatcgggatttgccattgtttccaaggataatgggatcagaataggttcaagagggcacatatttgttataagagagccctttgtatcttgtggcccttcagaatgtaggacctttttcttaacgcagggtgccttgctgaatgacaagcattcaaataacacagtaaaagacaggagtccctatcgggcattgatgagtgtaccattgggatcctctcctaatgcctaccaagccaaattcgagtccgtaggatggtctgcaacagcctgccatgatgggaaaaaatggatggccataggggtgagtggtgcagacgatgacgcttatgctgttattcattatgggggagtgccgacagatgtgataaggtcatggaggaagcaaatactaagaacacaagagtcttcgtgtgtttgcataaagggagagtgttattgggtaatgacggacggtccagcgaataatcaagcaagttacaaaatcttcaaatctcagaagggtatggtagttgatgaaaaagaaatttcattccaaggtgggcacattgaagaatgttcctgttatcccaacatggggaaagtggagtgtgtttgcagggacaattggaacggaatgaatagaccaattctgatattcgatgaaaagcttgagtatgaagtcggttatctatgcgctggaattccaacagacaccccaagagttcaagacagcagcttcactggctcatgcaccaatgcagttgggaggagtgggacaaacaattatggagtaaagggattcggctttagacaaggaaatagtgtatgggcgggaagaacaattagtgtttcttcacggagtggatttgaggttctattgatagaggacgggtggattagaccgagtaaaaccattagcaaaaaggttgaggttttgaacaataagaattggtcgggatacagcggggcttttaccatccccactgcaatgacaagcaagaactgtatagttccatgcttttggctggagatgattagggggaaaccagaggagaggactagcatttggacttcaagtagctccaccgtgttttgtggtgtttccagtgaggtcccaggatggtcgtgggatgatggagcaattcttccatttgacatcgataagatgtag

SEQ ID NO: 16 - DNA, 1413 bp, Influenza A virus
atgaatccaaatcagaagataatatgcatttcagccacaggaatgacactatcggtagtaagcctcctagtaggaatcgccaatttaggtctaaacatcggactccattataaagtaggtgatacaccaaatgtgaatattccaaacgtaaatgggaccaattcaacaacaacaataatcaacaacaacactcagaataatttcacaaatatcactaatattatacagagcaaaggtggagaaagaacatttctaaacctaactaagcccctatgtgaagtaaactcatggcacatcctatcaaaggacaatgcaataagaataggagaggatgctcatatcctggtcacaagagagccctatctatcttgcgatccacaagggtgcaggatgtttgctctaagtcaaggcacaacactcagagggcgacatgcaaatgggactatacatgataggagtccgttcagagcccttataagctgggaaatgggtcaagcacccagtccatataatactagggttgagtgcatagggtggtcaagcacatcatgccatgatggcatgtcaagaatgtcaatatgcatgtcaggaccgaacaacaatgcatcagcagtggtgtggtatgggggtaggccaataacagaaattccatcatgggcagggaatattctaagaacccaagaatcagagtgtgtgtgccataaaggagtttgtccagtagtcatgacagatggcccagcaaacaatagagcagcaaccaagataatctatttcaaggaagggaaaatacagaaaattgaagaactagcaggaaatgctcaacacattgaagaatgctcctgctatggagcaggaggagtgatcaaatgcatctgcagagacaattggaagggagcaaatagaccagtaatcactatagaccccgagatgatgacccatacaagcaagtatttatgctcaaaggtcctaactgatacgagtcgtcccaatgaccccactaatggaaactgtgacgcgccaataacaggagggagcccagatcctggagtcaaggggttcgcattcctagatggagaaaattcatggcttgggaggacaattagcaaagattccagatcaggttatgaaatgttaaaggtcccaaatgctgaaactgatatccaatcgggcccaatctcaaaccaggtgattgtcaacaaccaaaactggtcgggatactcaggggcattcatcgactactgggcaaacaaagaatgcttcaatccctgcttttatgtggaactaatcagagggaggcccaaagagagtagtgtactgtggacttcaaatagcatagtagctctctgtggatccaaaaagcgattaggatcatggtcctggcatgatggtgctgaaatcatctacttcgagtag

SEQ ID NO: 17 - DNA, 1410 bp, Influenza A virus
atgaatcctaatcagaaactctttgcatcatccggaatagcaatagcactaggaataataaatctcctaataggaatatccaatatgagtttaaatatatctctatactcaaaaggagaaaaccacaaaagtgataacctgacatgcacaaatatcaatcaaaacaataccaccatggtaaatacgtacatcaataacacaacaataattgacaaaaatacaaagatggagaaccctggttatctactgctgaataaaagtctatgcaacgttgaggggtgggttgtaatagcaaaggataatgcaattagatttggggaaagcgaacaaattatagtaactagagaaccctatgtctcatgtgatcctctaagttgtaaaatgtatgccctacatcaaggtacaacaatcagaaacaagcattcaaatggtaccacccacgatagaacagccttcagaggacttatctctaccccattaggcaaccccccaacagtgagcaacagtgagttcatatgtgttggatggtcaagcacaagctgccatgatggagtaagcagaatgacaatttgcgtacaaggaaacaatgaaaatgctactgcaacggtgtattacaacaagaggcttacaaccactattaaaacatgggctaaaaacattttaagaacccaagaatctgagtgtgtctgtcataacagtacttgtgtagtggtgatgactgatgggcccgcaaataaccaggcattcacaaaggtaatatactttcataaaggaacgataataaaagaagaaccactaaaaggttcagccaaacacatagaagaatgttcttgttatggtcataatcaaagagtgacttgtgtttgcagagacaattggcagggtgcaaatagacccgttatagaaattgacatgaataatttagaacacacaagtagatatatatgcacaggagtattaacagacaccagtagacccaaagataaagcaataggggaatgcttcaatcctattactgggagtcctggtgcaccaggaataaaaggtttcgggttcctaaatgagaataatacctggctggggagaacaatcagccccaaattgaggagtggatttgaaatgctgaagatacctaatgctgggactgacccagattccaaaataaaagaaagacaagaaatagttggtaatgacaattggtcaggctattccggaagtttcattgattattggaatgacaacagtgaatgctacaatccatgcttctatgtagaattaattagaggaaggcctgaagaagcaaaatatgttgaatggaccagtaacagcctaattgcactatgtgggagcccaatcccagttgggtctggatctttccctgatggggcacaaatcaaatacttttcgtaa

SEQ ID NO: 18 - DNA, 1413 bp, Influenza A virus
atgaatccaaatcagaaaataataaccattggctccatatcattgggattggttgtatttaatgttctactgcatgtcgtgagcatcatagtaacagtattagtcctgggaaaaggtggaaacaatggaatctgtaatgagacagtagtgagggaatacaacgagacagttaggatcgagaaggtaactcaatggcacaatactaatgtagtcgaatatgtgccgtattggaatgggggcacttacatgaataatacagaagcaatatgtgatgccaagggctttgcacctttttccaaggacaatggaataagaattggttccaggggacatatttttgttataagggagcccttcgtctcttgttcacctatagagtgcagaactttcttcctcacccaggggtccctgctcaatgacaaacactcaaatggaacagtgaaggataggagcccattcagaactctcatgagtgtcgaagtgggccaatcacccaatgtatatcaagccagatttgaagctgtggcgtggtcagcaacagcctgtcacgatgggaagaagtggatgacagttggtgtaacagggccagattccaaggcagtagcagtaattcactatggaggagtgcctactgatgtcgttaattcctgggcaggagatatattgaggactcaagagtcatcttgcacttgcatccaaggtgattgctattgggtaatgactgacggccccgccaataggcaggcacagtatagaatatacaaagcaaatcaaggcagaataattggccagacggatatcagttttaatggaggacatattgaagagtgttcttgctatcctaatgatggcaaagtggaatgtgtatgtagagacggttggactggaaccaacaggcctgtactagtcatctcgcctgatctctcttacagagttgggtacttatgtgcagggattcctagtgatactccaagaggggaagatacccaatttactggctcgtgcactagccccatggggaatcaggggtatggtgtaaaaggctttggatttcggcagggaactgatgtatggatggggcggacaattagtcgaacctccaggtcaggatttgagatattaaggataaagaatggttggacacagacaagtaaagagcagattagaaaacaggtagttgttgacaatttgaattggtcgggatacagtggatctttcactctgccagtagaattgtctgggaaagactgcctggttccctgtttttgggtagaaatgatcaggggcaagccagaagagaaaacaatctggacttccagcagttccattgtgatgtgtggagttgattatgaagttgccgattggtcatggcacgatggagctattcttccctttgacatcgataagatgtaa

SEQ ID NO: 19 - DNA, 1413 bp, Influenza A virus
atgaatccaaatcagaagattctatgcacttctgccactgctctcgtaataggcacaattgcagtactcataggaataacgaacttaggattgaacataggactacatctgaaaccgagctgcaattgctcacactcacaacccgaagcaaccaatgcaagccaaacaataataaataactattataatgacacaaacatcacccagataagtaataccaacattcaggtagaggaaagggcaattagagatttcaataacttgaccaaagggctctgtactataaattcatggcacatatatgggaaagacaatgcggtgagaattggggaggactcagatgttttagtcacaagagaaccctatgtctcctgtgacccagatgagtgcaggttctatgctctcagccaagggacaacaatcagaggaaaacactcaaatggaacaatacacgataggtctcaatatcgtgccctgataagctggccattgtcatcaccgcccacagtatacaacagcagagtggaatgcattggatggtcaagtactagttgtcatgatggcaaaaccaggatgtcaatatgcatatcaggcccgaacaataacgcatcagcagtgatctggtacaatagaaggcctgtgacagaaatcaacacatgggcccgaaacatactaaggacacaagaatctgaatgcgtatgccacaacggtgtctgcccggtagtgttcacagatgggtctgccactggacctgcagaaacaagaatatactattttaaagaagggaagatcttaaaatgggaacctctggctggaactgctaagcatatcgaagaatgctcatgctacggagagcgagcagagattacttgcacgtgtagggataattggcaaggctcaaatagaccagtaattcggatagatccagtggcgatgacacatactagtcagtatatatgtagccctgttctcacagataacccccgaccgaatgacccaactgtaggtaagtgtaacgacccttatccaggcaataacaacaatggggtcaaagggttttcatatctggatggagttaatacttggctagggaggacaataagcatagcttcaagatccggatatgagatgctaaaggtgccaaatgcattgacagacgataagtcaaagcccactcaaggtcagacaatcgtcttaaacactgactggagtggttacagtgggtccttcatggactattgggctgagggggaatgctaccgagcgtgtttttacgtggagttaatacgtgggagacctaaggaggataaagtgtggtggaccagtaatagtatagtatcgatgtgttccagcacagaattccttggacaatgggactggcctgatggggctaaaatagagtacttcctctaa

SEQ ID NO: 20 - DNA, 1557 bp, Influenza B virus
agcagaagcagagcatattcttagaactgaagtgaacaggccaaaaatgaacaatgctaccttcaactgtacaaacattaaccctattactcacatcagggggagtattattatcactatatgtgtcagcctcattgtcatacttattgtattcggatgtattgctaaaattttcatcaacaaaaacaactgcaccaacaatgtcattagagtgcacaaacgcatcaaatgcccagactgtgaaccattctgcaacaaaagagatgacatttccacccccagagccggagtggacataccctcgtttatcttgccagggctcaacctttcagaaggcactcctaattagccctcataggttcggagagatcaaaggaaactcagctcccttgataataagagaaccttttgttgcttgtggaccaaaagaatgcagacactttgctctgacccattatgcagctcagccggggggatactacaatggaacaagaaaggacagaaacaagctgaggcatctagtatcagtcaaattgggaaaaatcccaactgtggaaaactccattttccacatggcagcttggagcggatccgcatgccatgatggtagagaatggacatatatcggagttgatggtcctgacaatgatgcattggtcaaaataaaatatggagaagcatatactgacacatatcattcctatgcacacaacatcctaagaacacaagaaagtgcctgcaattgcatcgggggagattgttatcttatgataacagacggctcagcttcaggaattagtaaatgcagatttcttaaaattagagagggtcgaataataaaagaaatacttccaacaggaagagtggagcacactgaagagtgcacatgcgggttcgccagcaataaaaccatagaatgtgcctgtagagacaacagttacacagcaaaaagaccctttgtcaaattaaatgtggaaactgatacagctgaaataagattgatgtgcacaaagacttatctagacactcccagaccggatgatggaagcatagcagggccttgcgaatctaatggagacaagtggcttggaggcatcaaaggaggattcgtccatcaaagaatggcatctaagattggaagatggtactcccgaacgatgtctaaaactaacagaatggggatggaactgtatgtaaagtatgatggtgacccatggactgacagtgatgctcttactcttagtggagtaatggtttccatagaagaacctggttggtattcttttggcttcgaaataaaggacaagaaatgtgatgtcccttgtattgggatagagatggtacacgatggtggaaaagatacttggcattcagctgcaacagccatttactgtttgatgggctcaggacaattgctatgggacactgtcacaggcgttgatatggctttataatagaggaatggttggatctgttctaaaccctttgttcctattttatttgaacagttgttcttactagatttaattgtttctgaaaaatgctcttgttactact

SEQ ID NO: 21 - DNA, 655 bp, Artificial Sequence
  CMV promoter
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt